(19) United States
(12) Patent Application Publication
(10) Pub. No.: US 2002/0132793 A1
(43) Pub. Date: Sep. 19, 2002

(54) Use of methylphenidate compounds to enhance memory

(76) Inventors: Mel Epstein, Bristol, RI (US); Kjesten A. Wiig, Providence, RI (US)

Correspondence Address: ROPES & GRAY, ONE INTERNATIONAL PLACE, BOSTON, MA, 02110-2624, US

(21) Appl. No.: 10/087,232

(22) Filed: Feb. 28, 2002

Related U.S. Application Data
(63) Continuation-in-part of application No. 09/941,238, filed on Aug. 28, 2001.
(60) Provisional application No. 60/228,478, filed on Aug. 28, 2000.
(60) Provisional application No. 60/235,972, filed on Sep. 28, 2000.

(51) Int. Cl. A61K 31/675; A61K 31/445; G06F 17/60

(52) U.S. Cl. 514/79; 514/317; 705/2

(57) ABSTRACT

The present invention makes available methods and reagents for facilitating memory, e.g., to increase memory function such as long-term memory and recall ability. Memory is increased by increasing attention and long term consolidation.

### REFERENCE TO RELATED APPLICATIONS

This application is a continuation-in-part of U.S. Ser. No. 09/941,238 filed Aug. 28, 2001, and also claims priority to U.S. Provisional Patent Application No. 60/228,478 filed on Aug. 28, 2000 and to U.S. Provisional Patent Application No. 60/235,972 filed on Sep. 28, 2000, the specifications of each of which are incorporated by reference herein.

### BACKGROUND OF THE INVENTION

The term “memory” subsumes many different processes and requires the function of many different brain areas. Overall, human memory provides declarative recall, e.g., for facts and events accessible to conscious recollection, and non-declarative recall, e.g., procedural memory of skills and operations not stored regarding time and place. Research in recent years has provided information necessary to many of the various components of memory and identify associated brain regions. A newly acquired experience initially is susceptible to various forms of disruption. With time, however, the new experience becomes resistant to disruption. This observation has been interpreted to indicate that a labile, working, short-term memory is consolidated into a more stable, long-term memory.

Behavioral research has found that the human mind consolidates memory at certain key time intervals. The initial phase of memory consolidation occurs in the first few minutes after we are exposed to a new idea or learning experience. The next phase occurs over a longer period of time, such as during sleep. If a learning experience has on-going meaning to us, the next week or so serves as a further period of memory consolidation. In effect, in this phase, the memory moves from short-term to long-term storage.

Moreover, various mechanisms have been proposed to account for the formation of long-term memory. A wide range of observations suggest an evolutionarily conserved molecular mechanism involved with the formation of long-term memory. These include increased release of synaptic transmitter, increased number of synaptic receptors, decreased Km of receptors, synthesis of new memory factors either in the presynaptic or postsynaptic element, sprouting of new synaptic connections, increase of the active area in the presynaptic membrane and many others. Synaptic plasticity, the change in the strength of neuronal connections in the brain, is thought to underlie long-term memory storage.

“Memory consolidation”, or long-term memory is also believed to play a crucial role in a variety of neurological and mental disorders, including mental retardation, Alzheimer's disease and depression. Indeed, loss or impairment of long-term memory is significant feature of such diseases, and no effective therapy for that effect has emerged. Short-term, or “working” memory, is generally not significantly impaired in such patients.

It is, accordingly, an object of the present invention to provide methods and compositions for enhancing long-term memory function and/or performance. It is also an object of the present invention to provide methods and compositions for prophylactically (e.g., as a neuroprotective treatment) preventing or slowing degradation of long-term memory function and/or performance. It is also an object of the present invention to provide methods and compositions for restoring long-term memory function and/or performance.

### BRIEF SUMMARY OF THE INVENTION

One aspect of the present invention provides a method for enhancing memory, particularly long term memory, comprising administering to the animal a formulation of a methylphenidate compound, or pharmaceutically acceptable derivative, salt, solvate, pro-drug or metabolic derivative thereof, in an amount sufficient to enhance long-term memory in the animal. In such embodiments, the formulation includes at least 51 percent (w/w), 60 percent (w/w), 75 percent (w/w), 95 percent (w/w), or at least 99 percent (w/w) of the eutomers relative to the distomers of the methylphenidate compound(s).

In certain preferred embodiments, the formulation includes at least 51 percent (w/w), 60 percent (w/w), 75 percent (w/w), 95 percent (w/w), or at least 99 percent (w/w) of the eutomer(s) relative to the distomer(s) of the methylphenidate compound(s) represented by the general formula (I):

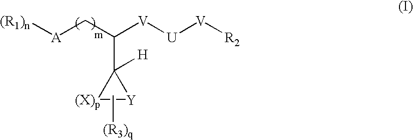

wherein

A represents a carbocyclic, heterocyclic, aryl, or heteroaryl ring;

U is absent (e.g., represents a bond between each V) or represents —C(═O)—, —C(═S)—, —P(═O)(OR₈)—, —S(O₂)—, or —S(O)—;

V, independently for each occurrence, is absent (represents a bond) or represents NR, O, or S;

Y represents NR₄, O, or S;

each occurrence of X, independently, is an atom selected from C, N, S, Se, and O;

R, independently for each occurrence, represents H, lower alkyl, lower alkenyl, aryl, heteroaryl, aralkyl, or heteroaralkyl;

each occurrence of R₁ represents, independently, aryl, C1-C6 alkyl, C1-C6 alkoxy, C1-C6 acyloxy, hydroxyl, C1-C6 alkanoyl, halogen, cyano, carboxyl, amido, amino, C1-C6 acylamino, C1-C6 alkylamino, nitro, sulfonic acid, or sulfhydryl;

R₂ is selected from H, C1-C6 alkyl, and C1-C6 alkanoyl;

R₃ represents, independently for each occurrence, hydrogen, C1-C6 alkyl, C1-C6 alkoxy, hydroxyl, C1-C6 alkanoyl, halogen, carboxyl, C2-C6 alkanoxy, nitro, or sulfhydryl, or two of R₃, taken together, represent an oxo group or a double bond between two adjacent X atoms;

R₄ represents hydrogen, lower alkyl, acyl, aryl, aralkyl, heteroaryl, or heteroaralkyl, preferably hydrogen or lower alkyl;

R₈ represents hydrogen, or a substituted or unsubstituted alkyl, alkenyl, aryl, aralkyl, cycloalkyl, cycloalkenyl, or heterocycle;

m is an integer selected from 0 and 1;

n is an integer from 0 to 7, preferably from 0 to 3;

p is an integer selected from 3, 4, 5, and 6; and

q is an integer from 0 to 16, preferably from 0 to 6; or

a pharmaceutically acceptable derivative, salt, solvate, pro-drug or metabolic derivative thereof.

Another aspect of the invention provides a method for enhancing memory consolidation in an animal, comprising administering to the animal a formulation of a methylphenidate compound, or pharmaceutically acceptable derivative, salt, solvate, pro-drug or metabolic derivative thereof, in an amount sufficient to enhance long-term memory in the animal, wherein the formulation includes at least 51 percent (w/w), 60 percent (w/w), 75 percent (w/w), 95 percent (w/w), or at least 99 percent (w/w) of the eutomer(s) relative to the distomer(s) of the methylphenidate compound.

In certain embodiments, the subject method utilizes a eutomer of the methylphenidate compound represented in the general formula (II), or pharmaceutically acceptable salt, pro-drug or metabolic derivative thereof:

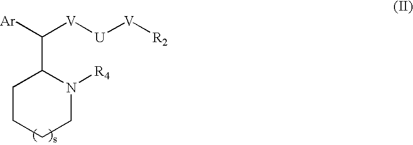

wherein

U is absent (represents a bond), or represents —C(═O)—, —C(═S)—, —P(═O)(OR₈)—, —S(O₂)—, or —S(O)—;

V, independently for each occurrence, is absent (represents a bond) or represents NR, O, or S;

R, independently for each occurrence, represents H, lower alkyl, lower alkenyl, aryl, heteroaryl, aralkyl, or heteroaralkyl;

R₂ is selected from H, C1-C6 alkyl, and C1-C6 alkanoyl;

R₄ represents hydrogen, lower alkyl, acyl, aryl, aralkyl, heteroaryl, or heteroaralkyl, preferably hydrogen or lower alkyl;

R₈ represents hydrogen, or a substituted or unsubstituted alkyl, alkenyl, aryl, aralkyl, cycloalkyl, cycloalkenyl, or heterocycle;

s represents an integer from 0 to 2; and

Ar represents a substituted or unsubstituted aryl or heteroaryl group.

In certain preferred embodiments, the subject method can use the pharmaceutically acceptable salt of a eutomer of the methylphenidate compound, and has a structure represented in the general formula (III):

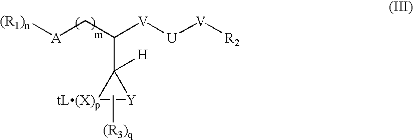

wherein

A represents a carbocyclic, heterocyclic, aryl, or heteroaryl ring;

U is absent (represents a bond) or represents —C(═O)—, —C(═S)—, —P(═O)(OR₈)—, —S(O₂)—, or —S(O)—;

V, independently for each occurrence, is absent (represents a bond) or represents NR, O, or S;

Y represents NR₄, O, or S;

each occurrence of X, independently, is an atom selected from C, N, S, Se, and O;

R, independently for each occurrence, represents H, lower alkyl, lower alkenyl, aryl, heteroaryl, aralkyl, or heteroaralkyl; each occurrence of R₁ represents, independently, aryl, C1-C6 alkyl, C1-C6 alkoxy, C1-C6 acyloxy, hydroxyl, C1-C6 alkanoyl, halogen, cyano, carboxyl, amido, amino, C1-C6 acylamino, C1-C6 alkylamino, nitro, sulfonic acid, or sulfhydryl;

R₂ is selected from H, C1-C6 alkyl, and C1-C6 alkanoyl;

R₃ represents, independently for each occurrence, hydrogen, C1-C6 alkyl, C1-C6 alkoxy, hydroxyl, C1-C6 alkanoyl, halogen, carboxyl, C2-C6 alkanoxy, nitro, or sulfhydryl, or two of R₃, taken together, represent an oxo group or a double bond between two adjacent X atoms;

R₄ represents hydrogen, lower alkyl, acyl, aryl, aralkyl, heteroaryl, or heteroaralkyl, preferably hydrogen or lower alkyl;

R₈ represents hydrogen, or a substituted or unsubstituted alkyl, alkenyl, aryl, aralkyl, cycloalkyl, cycloalkenyl, or heterocycle;

m is an integer selected from 0 and 1;

n is an integer from 0 to 7, preferably from 0 to 3;

p is an integer selected from 3, 4, 5, and 6; and

q is an integer from 0 to 16, preferably from 0 to 6;

L is a non-toxic organic or inorganic acid, or a quatemizing agent, or any combination thereof, and

t is an integer from 1 to 6, preferably from 1 to 2.

In other preferred embodiments, the subject method uses a pharmaceutically acceptable salt of a eutomer of the methylphenidate compound represented in the general formula (IV), or a pharmaceutically acceptable salt, solvate or pro-drug thereof:

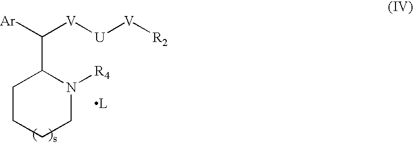

wherein

U is absent (represents a bond) or represents —C(═O)—, —C(═S)—, —P(═O)(OR₈)—,—S(O₂)—, or —S(O)—;

V, independently for each occurrence, is absent (represents a bond) or represents NR, O, or S;

R, independently for each occurrence, represents H, lower alkyl, lower alkenyl, aryl, heteroaryl, aralkyl, or heteroaralkyl;

R₂ is selected from H, C1-C6 alkyl, and C1-C6 alkanoyl;

R₄ represents hydrogen, lower alkyl, acyl, aryl, aralkyl, heteroaryl, or heteroaralkyl, preferably hydrogen or lower alkyl;

R₈ represents hydrogen, or a substituted or unsubstituted alkyl, alkenyl, aryl, aralkyl, cycloalkyl, cycloalkenyl, or heterocycle;

s represents an integer from 0 to 2;

Ar represents a substituted or unsubstituted aryl or heteroaryl group; and

L is a non-toxic organic or inorganic acid.

In still other preferred embodiments, the method is practiced with a metabolite of a eutomer of methylphenidate compound(s), e.g. using a compound represented in the general formula (V), or a pharmaceutically acceptable salt, solvate or pro-drug thereof:

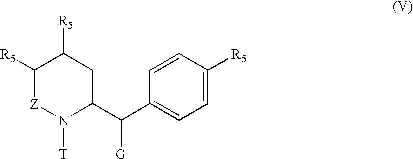

wherein

R₅, independently for each occurrence, is absent (represents a bond) or represents hydroxyl;

Z represents —CH₂— or —C(═O)—;

T represents hydrogen or —C(═O)—NH₂; G represents carboxylic acid, or a pharmaceutically acceptable salt thereof, carboxylic acid methyl ester, carboxylic acid ethyl ester, or acetylamino ethane sulfonic acid.

In certain embodiments, the pharmaceutical composition features one or more methylphenidate compounds provided in an amount sufficient to enhance long-term memory in a patient by a statistically significant amount when assessed by a standardized performance test. In preferred embodiments, the pharmaceutical composition features one or more methylphenidate compound(s) provided in an amount sufficient to enhance long-term memory in a patient by a statistically significant amount when assessed by one or more of a Cambridge Neuropsychological Test Automated Battery (CANTAB); a Children's Memory Scale (CMS); a Contextual Memory Test; a Continuous Recognition Memory Test (CMRT); a Denman Neuropsychology Memory Scale; a Full Object Memory Evaluation (FOME); a Graham-Kendall Memory for Designs Test; a Guild Memory Test; a Learning and Memory Battery (LAMB); a Memory Assessment Clinic Self-Rating Scale (MAC-S); a Memory Assessment Scales (MAS); a Randt Memory Test; a Recognition Memory Test (RMT); Rey Auditory and Verbal learning Test (RAVLT); Brief Visuospatial Memory Test (BVMT); Providence Recognition Memory Test (PRMT), a Rivermead Behavioral Memory Test; a Russell's Version of the Wechsler Memory Scale (RWMS); a Test of Memory and Learning (TOMAL); a Vermont Memory Scale (VMS); a Wechsler Memory Scale; and a Wide Range Assessment of Memory and Learning (WRAML). In certain embodiments, the pharmaceutical composition features one or more methylphenidate compounds provided in an amount sufficient to enhance long-term memory in a patient by a statistically significant amount when assessed by a Providence Recognition Memory Test.

Another aspect of the present invention relates to pharmaceutical preparations comprising a methylphenidate compound in an amount sufficient to enhance long-term memory in the animal. In certain embodiments, the preparation includes at least 51 mol percent, 60 mol percent, 75 mol percent, 95 mol percent, or at least 99 mol percent of the eutomer(s) with respect to the distomer(s) of the methylphenidate compound(s).

In certain embodiments, the subject pharmaceutical preparations are formulated for variable dosing, and preferably to deliver a sustained and increasing dose, e.g., over at least 4 hours, and more preferably over at least 8 or even 16 hours. For instance, the methylphenidate compound is contained within a nonabsorbable shell that releases the drug at a controlled rate.

Another aspect of the invention provides a kit comprising a methylphenidate compound formulation, e.g., as described herein and preferably provided in single dosage form or as a transdermal patch for enhancing memory in a patient (preferably a human), an in association with instructions (written and/or pictorial) describing the use of the formulation for enhancing memory, and optionally, warnings of possible side effects and drug-drug or drug-food interactions.

Yet another aspect of the invention relates to a method for conducting a pharmaceutical business, which includes:

a. manufacturing one or more of the subject preparations of methylphenidate; and

b. marketing to healthcare providers the benefits of using the preparation to increase memory function.

In certain embodiments, the subject business method can include providing a distribution network for selling the preparation. It may also include providing instruction material to patients or physicians for using the preparation to increase memory function.

Yet another aspect relates to a method for conducting a pharmaceutical business, including:

a. determining an appropriate preparation and dosage of a methylphenidate compound to increase memory function;

b. conducting therapeutic profiling of preparations identified in step (a), for efficacy and toxicity in animals; and

c. providing a distribution network for selling a preparation identified in step (b) as having an acceptable therapeutic profile.

For instance, the subject business method can include an additional step of providing a sales group for marketing the preparation to healthcare providers.

Still another aspect of the invention relates to a method for conducting a pharmaceutical business, including:

a. determining an appropriate preparation and dosage of methylphenidate to be administered to increase memory function; and

b. licensing, to a third party, the rights for further development and sale of the preparation.

### BRIEF DESCRIPTION OF THE DRAWINGS

FIG. 1 presents the effectiveness of various doses of methylphenidate on latency in passive avoidance testing, an indicator of memory consolidation.

FIG. 2 demonstrates the effect of methylphenidate on latency in passive avoidance testing.

FIG. 3 depicts the effects of methylphenidate on normal and fornix-lesioned animals.

FIGS. 4, 5, 6 and 7 show the effects of d and l-threo Methylphenidate on Inhibitory Avoidance.

FIGS. 8 and 9 show the effects of d and l-threo Methylphenidate on Activity Levels.

FIG. 10 shows an exemplary sustained release device.

FIG. 11 is a schematic illustration of a study design.

FIG. 12 shows memory retention at 60 minutes during ascending blood concentration of l-threo-methylphenidate.

FIG. 13 shows the consequence of l-threo-methylphenidate on patient scoring using the Rey Auditory & Visual Learning Test (30 minutes).

FIG. 14 shows the results of l-threo-methylphenidate on attention and learning as measured by the Brief Visuospatial Memory Test during ascending blood concentration of l-Threo-Methylphenidate.

### DETAILED DESCRIPTION OF THE INVENTION

I. Overview

The present invention relates to the discovery that the methylphenidate class of compounds (collectively referred to herein as “methylphenidate compounds”) can be used to enhance and/or restore long-term memory function and performance, e.g., to improve long-term memory (LTM) in animal subjects. More particularly, the invention relates to the discovery that particular stereoisomers of methylphenidate and related compounds and metabolites are the most effective for therapeutic use.

Furthermore, the present invention relates to the discovery that the methylphenidate compounds can be used to enhance and/or restore attention span and/or focus in animal subjects. The compounds can be useful in improving the attention span of normal individuals, as well as improving the attention span of individuals characterized by a deficit in attention span and/or focus (eg, individuals diagnosed with an attention deficit disorder). Lack of attentiveness may lead to a failure to process new information and accordingly commit such new information to memory. Lack of focus may also lead to difficulties in later recalling previously processed information. Thus, deficits in attentiveness and/or focus may affect learning and memory. In addition to memory and learning difficulties, lack of attentiveness has many other negative social and behavioral consequences. Accordingly, the subject methylphenidate compounds may be used to enhance and/or restore at least one of memory, learning, attentiveness, or focus.

Methylphenidate is a mild central nervous system stimulant. Its mode of action in humans is not fully understood, but presumably involves activation of the brain stem arousal system to effect stimulation of the patient. Methylphenidate is the most commonly prescribed psychotropic medication for children in the United States. It is used primarily for the treatment of children diagnosed with attention deficit disorder (ADD), and has a remarkable calming effect on these children, apparently unrelated to its memory-stimulating activity. Methylphenidate is synonymous with methyl α-phenyl-2-piperidineacetate, α-phenyl-2-piperidineacetate methyl ester, phenyl-piperidin-2-yl-acetic acid methyl ester, and methyl phenidylacetate. Methylphenidate is sold, in the form of the hydrochloride salt, as the product Ritalin™ and its generic equivalents. A comprehensive description of the compound can be found, for example, in Padmanabhan (1981, Analytical Profiles of Drug Substances v. 10, Florey, Ed., Academic Press, New York). Dosing and administration information, contraindications, warnings, and precautions pertaining to administration of methylphenidate to humans are available in the art (e.g., Physician's Desk Reference Registered TM, Medical Economics Co., Inc., Montvale, N.J., 51st ed., 1997; PDR Registered TM Generics TM, Medical Economics Co., Inc., Montvale, N.J., 2nd ed., 1996). Again, the term “methylphenidate compounds” is intended to include analogs of methylohenidate itself, include prodrug forms and metabolic derivatives.

However, racemic methylphenidate has many deleterious side-effects including insomnia, palpitation, headache, dyskinesia, drowsiness, tachycardia, angina, cardiac arrhythmia, abdominal pain, hypersensitivity (including skin rash, urticaria, fever, arthralgia, exfoliative dermatitis, erythema multiform with histopathological findings of necrotizing vasculitis, and thrombocytopenic purpura), anorexia, appetite suppression, irritability, attentional “sticking”, dizziness and dysphoria, increased aggression, and stunted growth. Additionally, racemic methylphenidate produces a euphoric effect when administered intravenously or through inhalation, and thus carries a potential for substance abuse in patients.

The present invention is based on utilizing a methylphenidate composition which is enriched for eutomers of methylphenidate compounds or pharmaceutically acceptable derivative, salt, solvate, clathrate, pro-drug or metabolic derivative thereof (collectively referred to herein as “methylphenidate compounds”) for increasing long-term potentiation and/or improving long-term memory in animals, such as humans. In certain embodiments, the methylphenidate mixture may be enriched for both L-threo (2S:2′S) and D-threo (2R:2′R) methylphenidate, e.g., comprising at least 60 by weight of these isomers, or more preferably at least 75, 90, 95 or even 99 percent by weight, relative to erythro isomers of the methylphendiate compound. Furthermore, the present invention is based on using the subject compounds for enhancing or restoring attention span and/or focus. The effects of the subject compounds on attention span may have secondary consequences on the ability to process and/or recall information, and therefore may also enhance memory and/or learning.

In certain preferred embodiments, the pharmaceutical preparations are enriched for a single enantiomer, such as the L-threo (2S:2′S) or D-threo (2R:2′R), and even more preferably is enriched for the L-threo (2S:2′S) enantiomer of methylphenidate. In preferred embodiments, methylphenidate provided in the formulation is at least 60 percent of the L-threo (2S:2′S) stereoisomer by weight relative to other isomers of methylphenidate, and more preferably at least 75, 90, 95 or even 99 percent by weight, relative to other stereoisomers of the methylphenidate, and particularly relative to the D-threo isomer.

The formulation includes a therapeutic amount of the methylphenidate compound necessary to affect memory enhancement, and, in preferred embodiments, has an appropriate ratio of eutomers to distomers to reduce at least a portion of the side effects of racemic methylphenidate.

II. Definitions

For convenience, certain terms employed in the specification, examples, and appended claims are collected here.

As used herein, “L-threo-methylphenidate” means the compound having the following formula:

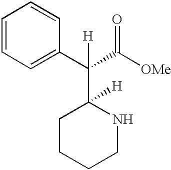

As used herein, “D-threo-methylphenidate” means the compound having the following formula:

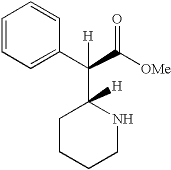

The term “ED₅₀” means the dose of a drug which produces 50% of its maximum response or effect.

An “effective amount” of, e.g., a methylphenidate compound, with respect to the subject method of treatment, refers to an amount of the activator in a preparation which, when applied as part of a desired dosage regimen brings about enhanced LTM according to clinically acceptable standards.

The term “LD₅₀” means the dose of a drug which is lethal in 50% of test subjects.

A “patient” or “subject” to be treated by the subject method can mean either a human or non-human animal.

The term “prodrug” is intended to encompass compounds which, under physiologic conditions, are converted into the therapeutically active agents of the present invention. A common method for making a prodrug is to include selected moieties which are hydrolyzed under physiologic conditions to reveal the desired molecule. In other embodiments, the prodrug is converted by an enzymatic activity of the host animal.

The term “therapeutic index” refers to the therapeutic index of a drug defined as LD₅₀/ED₅₀.

By “transdermal patch”, is meant a system capable of delivery of a drug to a patient via the skin, or any suitable external surface, including mucosal membranes, such as those found inside the mouth. Such delivery systems generally comprise a flexible backing, an adhesive and a drug retaining matrix, the backing protecting the adhesive and matrix and the adhesive holding the whole on the skin of the patient. On contact with the skin, the drug-retaining matrix delivers drug to the skin, the drug then passing through the skin into the patient's system.

Herein, the term “aliphatic group” refers to a straight-chain, branched-chain, or cyclic aliphatic hydrocarbon group and includes saturated and unsaturated aliphatic groups, such as an alkyl group, an alkenyl group, and an alkynyl group.

The terms “alkenyl” and “alkynyl” refer to unsaturated aliphatic groups analogous in length and possible substitution to the alkyls described above, but that contain at least one double or triple bond respectively.

The terms “alkoxyl” or “alkoxy” as used herein refers to an alkyl group, as defined above, having an oxygen radical attached thereto. Representative alkoxyl groups include methoxy, ethoxy, propyloxy, tert-butoxy and the like. An “ether” is two hydrocarbons covalently linked by an oxygen. Accordingly, the substituent of an alkyl that renders that alkyl an ether is or resembles an alkoxyl, such as can be represented by one of —O-alkyl, —O-alkenyl, —O-alkynyl, —O—(CH₂)ₘ—R_{8,} where R₈ represents an aryl, a cycloalkyl, a cycloalkenyl, a heterocycle or a polycycle; and m is zero or an integer in the range of 1 to 8.

The term “alkyl” refers to the radical of saturated aliphatic groups, including straight-chain alkyl groups, branched-chain alkyl groups, cycloalkyl (alicyclic) groups, alkyl-substituted cycloalkyl groups, and cycloalkyl-substituted alkyl groups. In preferred embodiments, a straight chain or branched chain alkyl has 30 or fewer carbon atoms in its backbone (e.g., C₁-C₃₀ for straight chains, C₃-C₃₀ for branched chains), and more preferably 20 or fewer. Likewise, preferred cycloalkyls have from 3-10 carbon atoms in their ring structure, and more preferably have 5, 6 or 7 carbons in the ring structure.

Moreover, the term “alkyl” (or “lower alkyl”) as used throughout the specification, examples, and claims is intended to include both “unsubstituted alkyls” and “substituted alkyls”, the latter of which refers to alkyl moieties having substituents replacing a hydrogen on one or more carbons of the hydrocarbon backbone. Such substituents can include, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxyl, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaromatic moiety. It will be understood by those skilled in the art that the moieties substituted on the hydrocarbon chain can themselves be substituted, if appropriate. For instance, the substituents of a substituted alkyl may include substituted and unsubstituted forms of amino, azido, imino, amido, phosphoryl (including phosphonate and phosphinate), sulfonyl (including sulfate, sulfonamido, sulfamoyl and sulfonate), and silyl groups, as well as ethers, alkylthios, carbonyls (including ketones, aldehydes, carboxylates, and esters), —CF₃, —CN and the like. Exemplary substituted alkyls are described below. Cycloalkyls can be further substituted with alkyls, alkenyls, alkoxys, alkylthios, aminoalkyls, carbonyl-substituted alkyls, —CF₃, —CN, and the like.

Unless the number of carbons is otherwise specified, “lower alkyl” as used herein means an alkyl group, as defined above, but having from one to ten carbons, more preferably from one to six carbon atoms in its backbone structure. Likewise, “lower alkenyl” and “lower alkynyl” have similar chain lengths. Throughout the application, preferred alkyl groups are lower alkyls. In preferred embodiments, a substituent designated herein as alkyl is a lower alkyl.

The term “alkylthio” refers to an alkyl group, as defined above, having a sulfur radical attached thereto. In preferred embodiments, the “alkylthio” moiety is represented by one of—S-alkyl, —S-alkenyl, —S-alkynyl, and —S—(CH₂)ₘ—R₈, wherein R₈ represents an aryl, a cycloalkyl, a cycloalkenyl, a heterocycle or a polycycle; and m is zero or an integer in the range of 1 to 8. Representative alkylthio groups include methylthio, ethylthio, and the like.

The terms “amine” and “amino” are art-recognized and refer to both unsubstituted and substituted amines, e.g., a moiety that can be represented by the

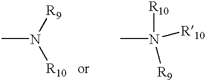

general formula:

wherein R₉, R₁₀ and R′₁₀ each independently represent a hydrogen, an alkyl, an alkenyl, —(CH₂)ₘ—R₈, or R₉ and R₁₀ taken together with the N atom to which they are attached complete a heterocycle having from 4 to 8 atoms in the ring structure; R₈ represents an aryl, a cycloalkyl, a cycloalkenyl, a heterocycle or a polycycle; and m is zero or an integer in the range of 1 to 8. In preferred embodiments, only one of R₉ or R₁₀ can be a carbonyl, e.g., R₉, R₁₀ and the nitrogen together do not form an imide. In even more preferred embodiments, R₉ and R₁₀ (and optionally R₁₀) each independently represent a hydrogen, an alkyl, an alkenyl, or —(CH₂)ₘ—R₈. Thus, the term “alkylamine” as used herein means an amine group, as defined above, having a substituted or unsubstituted alkyl attached thereto, i.e., at least one of R₉ and R₁₀ is an alkyl group.

The term “amido” is art-recognized as an amino-substituted carbonyl and includes a moiety that can be represented by the general formula:

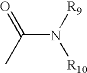

wherein R₉, R₁₀ are as defined above. Preferred embodiments of the amide will not include imides which may be unstable.

The term “aralkyl”, as used herein, refers to an alkyl group substituted with an aryl group (e.g., an aromatic or heteroaromatic group).

The term “aryl” as used herein includes 5-, 6-, and 7-membered single-ring aromatic groups that may include from zero to four heteroatoms, for example, benzene, pyrrole, fuiran, thiophene, imidazole, oxazole, thiazole, triazole, pyrazole, pyridine, pyrazine, pyridazine and pyrimidine, and the like. Those aryl groups having heteroatoms in the ring structure may also be referred to as “aryl heterocycles”, “heteroaryls”, or “heteroaromatics.” The aromatic ring can be substituted at one or more ring positions with such substituents as described above, for example, halogen, azide, alkyl, aralkyl, alkenyl, alkynyl, cycloalkyl, hydroxyl, alkoxyl, amino, nitro, sulfhlydryl, imino, amido, phosphate, phosphonate, phosphinate, carbonyl, carboxyl, silyl, ether, alkylthio, sulfonyl, sulfonamido, ketone, aldehyde, ester, heterocyclyl, aromatic or heteroaromatic moieties, —CF₃, —CN, or the like. The term “aryl” also includes polycyclic ring systems having two or more cyclic rings in which two or more carbons are common to two adjoining rings (the rings are “fused rings”) wherein at least one of the rings is aromatic, e.g., the other cyclic rings can be cycloalkyls, cycloalkenyls, cycloalkynyls, aryls and/or heterocyclyls.

The term “carbocycle” or “cyclic alkyl”, as used herein, refers to an aromatic or non-aromatic ring in which each atom of the ring is carbon.

The term “carbonyl” is art-recognized and includes such moieties as can be represented by the general formula:

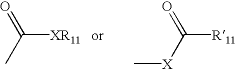

wherein X is a bond or represents an oxygen or a sulfur, and R₁₁ represents a hydrogen, an alkyl, an alkenyl, —(CH₂)ₘ—R₈ or a pharmaceutically acceptable salt, R′₁₁ represents a hydrogen, an alkyl, an alkenyl or —(CH₂)ₘ—R₈, where m and R₈ are as defined above. Where X is an oxygen and R₁₁ or R′₁₁ is not hydrogen, the formula represents an “ester”. Where X is an oxygen, and R₁₁ is as defined above, the moiety is referred to herein as a carboxyl group, and particularly when R₁₁ is a hydrogen, the formula represents a “carboxylic acid”. Where X is an oxygen, and R′₁₁ is hydrogen, the formula represents a “formate”. In general, where the oxygen atom of the above formula is replaced by sulfur, the formula represents a “thiocarbonyl” group. Where X is a sulfur and R₁₁ or R′₁₁ is not hydrogen, the formula represents a “thioester.” Where X is a sulfur and R₁₁ is hydrogen, the formula represents a “thiocatrboxylic acid.” Where X is a sulfur and R₁₁′ is hydrogen, the formula represents a “thioformate.” On the other hand, where X is a bond, and R₁₁ is not hydrogen, the above formula represents a “ketone” group. Where X is a bond, and R₁₁ is hydrogen, the above formula represents an “aldehyde” group.

The term “heteroatom” as used herein means an atom of any element other than carbon or hydrogen. Preferred heteroatoms are boron, nitrogen, oxygen, phosphorus, sulfur and selenium.

The terms “heterocyclyl” or “heterocyclic group” refer to 3- to 10-membered ring structures, more preferably 3- to 7-membered rings, whose ring structures include one to four heteroatoms. Heterocycles can also be polycycles. Heterocyclyl groups include, for example, thiophene, thianthrene, furan, pyran, isobenzofuran, chromene, xanthene, phenoxathiin, pyrrole, imidazole, pyrazole, isothiazole, isoxazole, pyridine, pyrazine, pyrimidine, pyridazine, indolizine, isoindole, indole, indazole, purine, quinolizine, isoquinoline, quinoline, phthalazine, naphthyridine, quinoxaline, quinazoline, cinnoline, pteridine, carbazole, carboline, phenanthridine, acridine, pyrimidine, phenanthroline, phenazine, phenarsazine, phenothiazine, furazan, phenoxazine, pyrrolidine, oxolane, thiolane, oxazole, piperidine, piperazine, morpholine, lactones, lactams such as azetidinones and pyrrolidinones, sultams, sultones, and the like. The heterocyclic ring can be substituted at one or more positions with such substituents as described above, as for example, halogen, alkyl, aralkyl, alkenyl, alkynyl, cycloalkyl, hydroxyl, amino, nitro, sulfhydryl, imino, amido, phosphate, phosphonate, phosphinate, carbonyl, carboxyl, silyl, ether, alkylthio, sulfonyl, ketone, aldehyde, ester, a heterocyclyl, an aromatic or heteroaromatic moiety, —CF₃, —CN, or the like.

The term “metabolites” refers to active derivatives produced upon introduction of a compound into a biological milieu, such as a patient.

As used herein, the term “nitro” means —NO₂; the term “halogen” designates—F, —Cl, —Br or —I; the term “sulfhydryl” means —SH; the term “hydroxyl” means —OH; and the term “sulfonyl” means —SO₂—.

The terms “polycyclyl” or “polycyclic group” refer to two or more rings (e.g., cycloalkyls, cycloalkenyls, cycloalkynyls, aryls and/or heterocyclyls) in which two or more carbons are common to two adjoining rings, e.g., the rings are “fused rings”. Rings that are joined through non-adjacent atoms are termed “bridged” rings. Each of the rings of the polycycle can be substituted with such substituents as described above, as for example, halogen, alkyl, aralkyl, alkenyl, alkynyl, cycloalkyl, hydroxyl, amino, nitro, sulfhydryl, imino, amido, phosphate, phosphonate, phosphinate, carbonyl, carboxyl, silyl, ether, alkylthio, sulfonyl, ketone, aldehyde, ester, a heterocyclyl, an aromatic or heteroaromatic moiety, —CF₃, —CN, or the like.

The phrase “protecting group” as used herein means temporary substituents which protect a potentially reactive functional group from undesired chemical transformations. Examples of such protecting groups include esters of carboxylic acids, silyl ethers of alcohols, and acetals and ketals of aldehydes and ketones, respectively. The field of protecting group chemistry has been reviewed (Greene, T. W.; Wuts, P. G. M. Protective Groups in Organic Synthesis, 2^{nd} ed.; Wiley: New York, 1991).

As used herein, the term “substituted” is contemplated to include all permissible substituents of organic compounds. In a broad aspect, the permissible substituents include acyclic and cyclic, branched and unbranched, carbocyclic and heterocyclic, aromatic and nonaromatic substituents of organic compounds. Illustrative substituents include, for example, those described herein above. The permissible substituents can be one or more and the same or different for appropriate organic compounds. For purposes of this invention, the heteroatoms such as nitrogen may have hydrogen substituents and/or any permissible substituents of organic compounds described herein which satisfy the valences of the heteroatoms. This invention is not intended to be limited in any manner by the permissible substituents of organic compounds.

It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc.

The term “sulfamoyl” is art-recognized and includes a moiety that can be represented by the general formula:

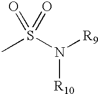

in which R₉ and R₁₀ are as defined above.

The term “sulfate” is art recognized and includes a moiety that can be represented by the general formula:

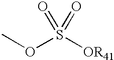

in which R₄₁ is an electron pair, hydrogen, alkyl, cycloalkyl, or aryl.

The term “sulfonamido” is art recognized and includes a moiety that can be represented by the general formula:

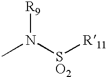

in which R₉ and R′₁₁ are as defined above.

The term “sulfonate” is art-recognized and includes a moiety that can be represented by the general formula:

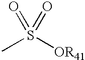

in which R₄₁ is an electron pair, hydrogen, alkyl, cycloalkyl, or aryl.

The terms “sulfoxido” or “sulfinyl”, as used herein, refers to a moiety that can be represented by the general formula:

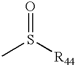

in which R₄₄ is selected from the group consisting of hydrogen, alkyl, alkenyl, alkynyl, cycloalkyl, heterocyclyl, aralkyl, or aryl.

The term “sulfonyl”, as used herein, refers to a moiety that can be represented by the general formula:

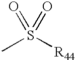

in which R₄₄ is selected from the group consisting of hydrogen, alkyl, alkenyl, alkynyl, cycloalkyl, heterocyclyl, aryl, or heteroaryl.

Analogous substitutions can be made to alkenyl and alkynyl groups to produce, for example, aminoalkenyls, aminoalkynyls, amidoalkenyls, amidoalkynyls, iminoalkenyls, iminoalkynyls, thioalkenyls, thioalkynyls, carbonyl-substituted alkenyls or alkynyls.

As used herein, the definition of each expression, e.g., alkyl, m, n, etc., when it occurs more than once in any structure, is intended to be independent of its definition elsewhere in the same structure.

Contemplated equivalents of the compounds described above include compounds which otherwise correspond thereto, and which have the same general properties thereof (e.g., the ability to effect long-term memory), wherein one or more simple variations of substituents are made which do not adversely affect the efficacy of the compound. In general, the compounds of the present invention may be prepared by the methods illustrated in the general reaction schemes as, for example, described below, or by modifications thereof, using readily available starting materials, reagents and conventional synthesis procedures. In these reactions, it is also possible to make use of variants which are in themselves known, but are not mentioned here.

For purposes of this invention, the chemical elements are identified in accordance with the Periodic Table of the Elements, CAS version, Handbook of Chemistry and Physics, 67th Ed., 1986-87, inside cover. Also for purposes of this invention, the term “hydrocarbon” is contemplated to include all permissible compounds having at least one hydrogen and one carbon atom. In a broad aspect, the permissible hydrocarbons include acyclic and cyclic, branched and unbranched, carbocyclic and heterocyclic, aromatic and nonaromatic organic compounds which can be substituted or unsubstituted.

III. Exemplary Compounds of the Invention

In certain embodiments, the methylphenidate compound is represented by the general formula (I), or pharmaceutically acceptable salt, pro-drug or metabolic derivative thereof:

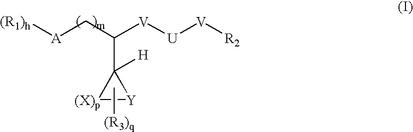

wherein

A represents a carbocyclic, heterocyclic, aryl, or heteroaryl ring;

U is absent (represents a bond) or represents —C(═O)—, —C(═S)—, —P(═O)(OR₈)—, —S(O₂)—, or —S(O)—;

V, independently for each occurrence, is absent (represents a bond) or represents NR, O, or S;

Y represents NR₄, O, or S, preferably NR₄;

each occurrence of X, independently, is an atom selected from C, N, S, Se, and O;

R, independently for each occurrence, represents H, lower alkyl, lower alkenyl, aryl, heteroaryl, aralkyl, or heteroaralkyl;

each occurrence of R₁ represents, independently, aryl, C1-C6 alkyl, C1-C6 alkoxy, C1-C6 acyloxy, hydroxyl, C1-C6 alkanoyl, halogen, cyano, carboxyl, amido, amino, C1-C6 acylamino, C1-C6 alkylamino, nitro, sulfonic acid, or sulfhydryl;

R₂ is selected from H, C1-C6 alkyl, and C1-C6 alkanoyl;

R₃ represents, independently for each occurrence, hydrogen, C1-C6 alkyl, C1-C6 alkoxy, hydroxyl, C1-C6 alkanoyl, halogen, carboxyl, C2-C6 alkanoxy, nitro, or sulfhydryl, or two of R₃, taken together, represent an oxo group or a double bond between two adjacent X atoms;

R₄ represents hydrogen, lower alkyl, acyl, amido, ester, aryl, aralkyl, heteroaryl, or heteroaralkyl, preferably hydrogen or lower alkyl;

R₈ represents hydrogen, or a substituted or unsubstituted alkyl, alkenyl, aryl, aralkyl, cycloalkyl, cycloalkenyl, or heterocycle;

m is an integer selected from 0 and 1; and

n is an integer from 0 to 7, preferably from 0 to 3;

p is an integer selected from 3, 4, 5, and 6; and

q is an integer from 0 to 16, preferably from 0 to 6.

The composition may include a mixture of the two isomeric forms.

In certain embodiments, a subject compound may have a structure represented by the general formula (II), or pharmaceutically acceptable salt or pro-drug thereof:

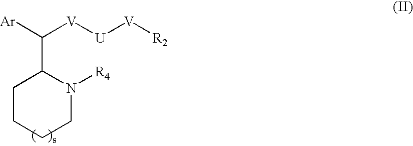

wherein U, V, R₄, R₂, and R are defined as above;

s represents an integer from 0 to 2; and

Ar represents a substituted or unsubstituted aryl or heteroaryl group.

In certain embodiments of Formula I or II, R₂ represents H or C1-C6 alkyl, preferably H or C1-C3 alkyl.

In certain embodiments of Formula I or II, U represents —C(═O)— or —C(═S)—, preferably —C(═O)—. In certain embodiments of Formula I or II, V represents NH, S, or O, preferably O. In certain embodiments of Formula I or II, at least one occurrence of V is present, and preferably U is present.

In certain embodiments of Formula I, m is 0.

In certain embodiments of Formula I, p is an integer from 3 to 5, preferably 4.

In certain embodiments of Formula I, A represents an aryl or heteroaryl group, preferably a phenyl group.

In certain embodiments of Formula I, R₃ represents, independently for each occurrence, H or C1-C3 alkyl in all occurrences, preferably H.

In certain embodiments of Formula I, R₁ represents, independently for each occurrence, H, halogen, C1-C6 alkyl, hydroxyl, nitro, or carboxyl.

As set out above, certain embodiments of compounds of formulae I and II may contain a basic functional group, such as amino or alkylamino, and thus, can be utilized in a free base form or as pharmaceutically acceptable salt forms derived from pharmaceutically acceptable organic and inorganic acids.

The pharmaceutically acceptable salts of the subject compounds I and II include the conventional non-toxic salts and/or quaternary ammonium salts of the compounds, e.g., from non-toxic organic or inorganic acids. For example, such conventional non-toxic salts include those derived from inorganic acids such as hydrochloride, hydrobromic, sulfuric, sulfamic, phosphoric, nitric, and the like; and the salts prepared from organic acids such as acetic, 2-acetoxybenzoic, ascorbic, benzene sulfonic, benzoic, chloroacetic, citric, ethane disulfonic, ethane sulfonic, formic, fumaric, gluconic, glutamic, glycolic, hydroxymaleic, isothionic, lactic, maleic, malic, methanesulfonic, oxalic, palmitic, phenylacetic, propionic, salicyclic, stearic, succinic, sulfanilic, tartaric, toluenesulfonic, and the like.

In certain embodiments, such salts have a structure represented by the general formula III:

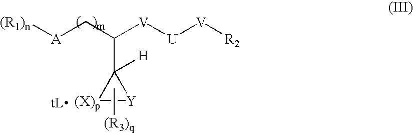

wherein R₁, n, A, m, V, u, R₂, X, p, R₃ and q are defined as above;

L is a non-toxic organic or inorganic acid, or a quaternizing agent, or any combination thereof, and

t is an integer from 1 to 6, preferably from 1 to 2.

In certain embodiments, L is selected from the following inorganic acids: hydrochloric, hydrobromic, nitric, phosphoric, sulfamic, and sulfuric, or from the following organic acids: 2-acetoxybenzoic, ascorbic, benzene sulfonic, benzoic, chloroacetic, citric, ethane disulfonic, ethane sulfonic, formic, fumaric, gluconic, glutamic, glycolic, hydroxymaleic, isothionic, lactic, maleic, malic, methanesulfonic, oxalic, palmitic, phenylacetic, propionic, salicyclic, stearic, succinic, sulfanilic, tartaric, and toluenesulfonic.

In certain embodiments, these salts have a structure represented by the general formula IV:

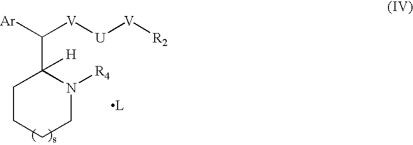

wherein U, V, R₄, R₂, R, Ar, s, and L are defined as above.

The subject methylphenidate compounds can also be provided as prodrugs.

The compounds of the present invention further include metabolites of the subject methylphenidate compound, including but not limited to the following: Phenyl-piperidin-2-yl-acetic acid, (4-Hydroxy-phenyl)-(piperidin-2-yl)-acetic acid methyl ester, (4-Hydroxy-phenyl)-(piperidin-2-yl)-acetic acid, (6-Oxo-piperidin-2-yl)-phenyl-acetic acid methyl ester, (6-Oxo-piperidin-2-yl)-phenyl-acetic acid, (4-Hydroxy-phenyl)-(6-oxo-piperidin-2-yl)-acetic acid methyl ester, 2-[2-(4-Hydroxy-phenyl)-2-(6-oxo-piperidin-2-yl)-acetylamino]-ethanesulfonic acid, (5-Hydroxy-6-oxo-piperidin-2-yl)-phenyl-acetic acid, (1-Carboamyl-piperidin-2-yl)-phenyl-acetic acid methyl ester, 1-Carboamoyl-piperidin-2-yl)-phenyl-acetic acid, (5-Hydroxy-6-oxo-piperidin-2-yl)-phenyl-acetic acid methyl ester, (4-Hydroxy-6-oxo-piperidin-2-yl)-phenyl-acetic acid methyl ester, 3,4,5-Trihydroxy-6-[2-(methoxycarbonyl-phenyl-methyl)-6-oxo-piperidin-4-yloxy]-tetrahydropyran-2-carboxylic acid, 3,4,5-Trihydroxy-6-{4-[methoxycarbonyl-(6-oxo-piperidin-2-yl)-methyl]-phenoxy}-tetrahydropyran-2-carboxylic acid, 6-[4-(Carboxy-piperidin-2-yl-methyl)-phenoxy]-3,4,5-trihydroxy-tetrahydro-pyran-2-carboxylic acid, 3,4,5-Trihydroxy-6-[6-(methoxycarbonyl-phenyl-methyl)-2-oxo-piperidin-3-yloxy]-tetrahydropyran-2-carboxylic acid, 3,4,5-Trihydroxy-6-[2-(6-oxo-piperidin-2-yl)-2-phenyl-acetoxy]-tetrahydro-pyran-2-carboxylic acid, and phenyl-piperidin-2-yl-acetic acid ethyl ester. In preferred embodiments, these metabolites are selected from the following compounds: Phenyl-piperidin-2-yl-acetic acid, (4-Hydroxy-phenyl)-(piperidin-2-yl)-acetic acid methyl ester, (4-Hydroxy-phenyl)-(piperidin-2-yl)-acetic acid, (6-Oxo-piperidin-2-yl)-phenyl-acetic acid methyl ester, and (6-Oxo-piperidin-2-yl)-phenyl-acetic acid.

In certain embodiments, the selected metabolite has a structure represented by the general formula V:

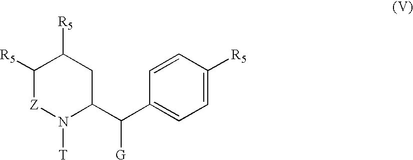

wherein

R₅, independently for each occurrence, is absent (represents a bond) or represents hydroxyl;

Z represents —CH₂— or —C(═O)—;

T represents hydrogen or —C(═O)—NH₂;

G represents carboxylic acid, or a pharmaceutically acceptable salt thereof, carboxylic acid methyl ester, carboxylic acid ethyl ester, carboxylic acid O-glucuronide, or acetylamino ethane sulfonic acid.

In certain embodiments, the method includes administering, conjointly with the pharmaceutical preparation, one or more of a neuronal growth factor, a neuronal survival factor, and a neuronal tropic factor. Additionally or alternatively, a subject compound may be administered in conjunction with a cholinergic, adrenergic, dopaminergic, or glutaminergic activator. An agent to be administered conjointly with a subject compound may be formulated together with a subject compound as a single pharmaceutical preparation, e.g., as a pill or other medicament including both agents, or may be administered as a separate pharmaceutical preparation.

In another aspect, the present invention provides pharmaceutical preparations comprising, as an active ingredient, a stereoisomerically enriched preparation in a eutomer of a methylphenidate compound(s), or derivatives thereof. The subject methylphenidate compound is formulated in an amount sufficient to improve LTP in an animal. The subject preparations and methods can be treatments using methylphenidate compounds effective for human and/or animal subjects. In addition to humans, other animal subjects to which the invention is applicable extend to both domestic animals and livestock, raised either as pets or for commercial purposes. Examples are dogs, cats, cattle, horses, sheep, hogs, and goats.

Still another aspect of the invention relates to the use of stereoisomerically pure preparations of methylphenidate compounds for lessening the severity or prophylactically preventing the occurrence of learning and/or memory defects in an animal, and thus, altering the learning ability and/or memory capacity of the animal. As a result, the compounds of the present invention may be useful for treating and/or preventing memory impairment, e.g., due to toxicant exposure, brain injury, brain aneurysm, age-associated memory impairment, mild cognitive impairment, epilepsy, mental retardation in children, and dementia resulting from a disease, such as Parkinson's disease, Alzheimer's disease, AIDS, head trauma, Huntington's disease, Pick's disease, Creutzfeldt-Jakob disease, Anterior Communicating Artery Syndrome, hypoxia, post cardiac surgery, Downs Syndrome and Stroke. In addition, the compounds of the invention may be useful in enhancing memory in normal individuals.

Still another aspect of the present invention relates to the use of stereoisomerically pure preparations of methylphenidate compounds for lessening the severity or prophylactically preventing the occurrence of deficits in attention span. As a result, the subject compounds may be useful for treating and/or preventing deficits in focus and/or attention span. Such defects in focus and/or attention span have a wide range of social, psychological, and behavioral consequences. Additionally, deficits in focus and/or attention span may have secondary consequences on memory and learning by affecting either the ability to commit new information to memory or the ability to recall previously processed information. The methylphenidate compounds of the present invention may be useful for treating any condition characterized in whole or in part by a deficit in attention span or focus. Examples of conditions which may be treated using the subject compounds include, but are not limited to, Attention Deficit Disorder (ADD), Attention Deficit Disorder with Hyperactivity (ADHD), autism, Tourette's syndrome, bi-polar disorder, and depression. In addition, the compounds may be useful in improving attention span and focus in normal individuals (i.e., individuals with no identified condition which impairs attention span).

The invention also relates to the conjoint use of a methylphenidate compound with agents that mimic or stimulate PKC and/or PKA pathways.

A. Synthesis of Methylphenidate compounds

As described in further detail below, it is contemplated that the subject methods can be carried out using a stereoisomerically enriched preparation in a eutomer of methylphenidate compound(s), e.g., L-threo-methylphenidate (2S:2′S), or of both L-threo (2S:2′S) and L-erythro (2S:2′R), both L-threo (2S:2′S) and D-erythro (2R:2′S), or of both L-threo (2S:2′S) and D-threo (2R:2′R), or a variety of different derivatives thereof. The suitability of use of a particular methylphenidate compound can be readily determined, for example, by such drug screening assays as described herein.

The subject methylphenidate compounds, and derivatives thereof, can be prepared readily by employing known synthetic methodology. As is well known in the art, these coupling reactions are carried out under relatively mild conditions and tolerate a wide range of “spectator” functionality. Additional compounds may be synthesized and tested in a combinatorial fashion, to facilitate the identification of additional methylphenidate compounds which may be employed in the subject method.

Numerous methods for synthesizing methylphenidate, for interconverting the diastereomers of methylphenidate, and for resolving the enantiomers of methylphenidate have been described in the art (U.S. Pat. No. 2,507,631 to Hartmann; U.S. Pat. No. 2,838,519 to Rometsch; U.S. Pat. No. 2,957,880 to Rometsch; British Patent Nos. 788,226 and 878,167, each to Ciba Ltd.; Soviet Patent No. 466,229 to Yakhontov et al.; International Patent Application Publication No. WO9735836 of Fox et al.; International Patent Application Publication No. WO9728124 of Langston et al.; Panizzon, 1944, Helv. Chim. Acta 27:1748-1756; Naito et al., 1964, Chem. Pharm. Bull. 12:588-590; Deutsch et al., 1996, J. Med. Chem. 39:1201-1209; Earle et al., 1969, J. Chem. Soc. (C) 2093-2098); International Patent Application Publication No. WO9825902 of Faulconbridge et al.; Patrick et al., 1987, J. Pharmacol. Exp. Therapeut. 241:152-158 International Patent Application Publication No. WO9727176 of Harris et al.; International Patent Application Publication No. WO9825902 of Zavareh. Other resolution processes are described in PCT/GB97/00185 and PCT/GB97/00643. Such resolutions may be combined with the racemization described in PCT/GB97/00281. The contents of these publications are incorporated herein by reference.

In one embodiment, a subject methylphenidate compound can be synthesized according to the methods set forth in U.S. Pat. No. 6,025,502. Briefly, a first compound having the formula (VI)

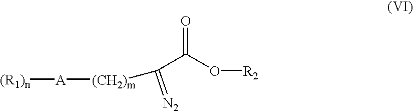

is combined with a second compound having the formula (VII)

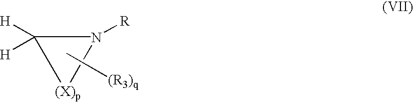

in the presence of a rhodium catalyst to form a reaction intermediate, and thereafter removing the nitrogen-protecting group (i.e., R in formula (VII)) from the reaction intermediate. The catalyst is preferably a dirhodium (II) tetrakis[methyl 2-oxopyrrolidine-5(R)-carboxylate] (herein “Rh₂[5R-MBPY]₄”) catalyst.

Each X in formula (VII) is preferably carbon, although heterocyclic rings comprising more than the nitrogen atom indicated in formula (VII) may also be used. Non-aryl compounds having formula (VII) are preferred in the methods of the invention. The nitrogen-protecting group may be any of a wide variety of nitrogen-protecting groups such as, for example, a butoxycarbonyl (“Boc”) group, a 9-fluorenylmethoxy-carbonyl (“Fmoc”) group, and the like. Methods of removing nitrogen-protecting groups are well known in the art. By way of example, it is known that Boc groups are acid labile, and may be removed by treatment with trifluoroacetic acid, and that Fmoc groups are base labile, and may be removed by treatment with piperidine. Suitable second compounds include, for example, N-Boc-piperidine, N-Boc-pyrrolidine, and N-Boc-pyridine.

Combining the first and second compounds in the presence of a rhodium catalyst leads to formation of a reaction intermediate in which the ratio of the L-enantiomer to the D-enantiomer is greater than 1.00, and is preferably greater than about 1.25 or 1.5, and in which the ratio of the threo-diastereomer to the erythro-diastereomer is greater than 1.00, and is preferably greater than about 2, 5, or 10.

Removal of the nitrogen-protecting (i.e., Z) group from this intermediate yields the methylphenidate derivative having the enantiomeric and diastereomeric ratios described above. When the nitrogen-protecting group is a Boc group, for example, it may be removed by maintaining the reaction intermediate in an acidic environment (e.g., in HCl-acidified methanol at 0° C.).

The compounds of the present invention, particularly libraries of methylphenidate analogs having various representative classes of substituents, are amenable to combinatorial chemistry and other parallel synthesis schemes (see, for example, PCT WO 94/08051). The result is that large libraries of related compounds, e.g., a variegated library of compounds represented above, can be screened rapidly in high throughput assays in order to identify potential methylphenidate analogs, as well as to refine the specificity, toxicity, and/or cytotoxic-kinetic profile of a lead compound.

Simply for illustration, a combinatorial library for the purposes of the present invention is a mixture of chemically related compounds which may be screened together for a desired property. The preparation of many related compounds in a single reaction greatly reduces and simplifies the number of screening processes which need to be carried out. Screening for the appropriate physical properties can be done by conventional methods.

Diversity in the library can be created at a variety of different levels. For instance, the substrate aryl groups used in the combinatorial reactions can be diverse in terms of the core aryl moiety, e.g., a variegation in terms of the ring structure, and/or can be varied with respect to the other substituents.

A variety of techniques are available in the art for generating combinatorial libraries of small organic molecules such as the subject methylphenidate compounds. See, for example, Blondelle et al. (1995) Trends Anal. Chem. 14:83; the Affymax U.S. Pat. Nos. 5,359,115 and 5,362,899: the Ellman U.S. Pat. No. 5,288,514: the Still et al. PCT publication WO 94/08051; the ArQule U.S. Pat. Nos. 5,736,412 and 5,712,171; Chen et al. (1994) JACS 116:2661: Kerr et al. (1993) JACS 115:252; PCT publications WO92/10092, WO93/09668 and WO91/07087; and the Lerner et al. PCT publication WO93/20242). Accordingly, a variety of libraries on the order of about 100 to 1,000,000 or more diversomers of the subject methylphenidate compounds can be synthesized and screened for particular activity or property.

In an exemplary embodiment, a library of candidate methylphenidate compound diversomers can be synthesized utilizing a scheme adapted to the techniques described in the Still et al. PCT publication WO 94/08051, e.g., being linked to a polymer bead by a hydrolyzable or photolyzable group, optionally located at one of the positions of the candidate regulators or a substituent of a synthetic intermediate. According to the Still et al. technique, the library is synthesized on a set of beads, each bead including a set of tags identifying the particular diversomer on that bead. The bead library can then be “plated” with cells for which a methylphenidate compound is sought. The diversomers can be released from the bead, e.g., by hydrolysis.

Many variations on the above and related pathways permit the synthesis of widely diverse libraries of compounds which may be tested as methylphenidate compounds.

B. Generation of Animal Models to Test Agents

Applicants have previously described an animal model for studying fornix-mediated memory consolidation. See, for example, Taubenfield et al., Supra. The fornix-lesioned animals can be used for drug screening, e.g., to identify dosages of the subject compositions which enhance memory consolidation. The lesioned mammal can have a lesion of the fornix or a related brain structure that disrupts memory consolidation (e.g., perirhinal cortex, amygdala, medial septal nucleus, locus coeruleus, hippocampus, mammillary bodies). Lesions in the mammal can be produced by mechanical or chemical disruption. For example, the fornix lesion can be caused by surgical ablation, electrolytic, neurotoxic and other chemical ablation techniques, or reversible inactivation such as by injection of an anesthetic, e.g., tetrodotoxin or lidocaine, to temporarily arrest activity in the fornix.

To further illustrate, fimbrio-fornix (rodents) and fornix (primates) lesions can be created by stereotactic ablation. In particular, neurons of the fornix structure are axotomized, e.g., by transection or aspiration (suction) ablation. A complete transection of the fornix disrupts adrenergic, cholinergic and GABAergic function and electrical activity, and induces morphological reorganization in the hippocampal formation. In general, the fornix transection utilized in the subject method will not disconnect the parahippocampal region from the neocortex. In those embodiments, the fornix transection will not disrupt functions that can be carried out by the parahippocampal region independent of processing by the hippocampal formation, and hence would not be expected to produce the full-blown amnesia seen following more complete hippocampal system damage.

In one embodiment, the animal can be a rat. Briefly, the animals are anesthetized, e.g., with intraperitoneal injections of a ketamine-xylazine mixture and positioned in a Kopf stereotaxic instrument. A sagittal incision is made in the scalp and a craniotomy is performed extending 2.0 mm posterior and 3.0 mm lateral from Bregma. An aspirative device, e.g., with a 20 gauge tip, is mounted to a stereotaxic frame (Kopf Instruments) and fimbria-fornix is aspirated by placing the suction tip at the correct sterotaxic location in the animals brain. Unilateral aspirative lesions are made by suction through the cingulate cortex, completely transecting the fimbria fornix unilaterally, and (optionally) removing the dorsal tip of the hippocampus as well as the overlying cingulate cortex to inflict a partial denervation on the hippocampus target. See also, Gage et al., (1983) Brain Res. 268:27 and Gage et al. (1986) Neuroscience 19:241.

In another exemplary embodiment, the animal can be a monkey. The animal can be anesthetized, e.g., with isoflurane (1.5-2.0%). Following pretreatment with mannitol (0.25 g/kg, iv), unilateral transections of the left fornix can be performed, such as described by Kordower et al. (1990) J. Comp. Neurol., 298:443. Briefly, a surgical drill is used to create a parasagittal bone flap which exposes the frontal superior sagittal sinus. The dura is retracted and a self-retaining retractor is used to expose the interhemispheric fissure. The corpus callosum is longitudinally incised. At the level of the foramen of Monro, the fornix is easily visualized as a discrete 2-3 mm wide white fiber bundle. The fornix can be initially transected using a ball dissector. The cut ends of the fornix can then be suctioned to ensure completeness of the lesion.

In still other illustrative embodiments, the fornix lesion can be created by excitotoxically, or by other chemical means, inhibiting or ablating fornix neurons, or the cells of the hippocampus which are innervated by fornix neurons. In certain preferred embodiments, the fornix lesion is generated by selective disruption of particular neuronal types, such as fornix cholinergic and adrenergic neurons.

For instance, the afferant fornix signals to the hippocampus due to cholinergic neurons can be ablated by atropine blockade. Another means for ablation of the cholinergic neurons is the use of 192IgG-saporin (192IgG-sap), e.g., intraventricularly injection into the fornix and hippocampus. The agents such as 6-OHDA and ibotenic acid can be used to selectively destroy fornix dopamine neurons as part of the ablative regimen.

In preferred embodiments, the animal is a non-human mammal, such as a dog, cat, horse, cow, pig, sheep, goat, chicken, monkey, ape, rat, rabbit, etc. In certain preferred embodiments, the animal is a non-human primate. In other preferred embodiment, the animal is a rodent.

There are a variety of tests for cognitive function, especially learning and memory testing, which can be carried our using the lesioned and normal animals. Learning and/or memory tests include, for example, inhibitory avoidance, contextual fear conditioning, visual delay non-match to sample, spatial delay non-match to sample, visual discrimination, Barnes circular maze, Morris water maze, and radial arm maze tests.

An exemplary passive avoidance test utilizes an apparatus that consists of a lit chamber that can be separated from a dark chamber by a sliding door. At training, the animal is placed in the lit chamber for some period of time, and the door is opened. The animal moves to the dark chamber after a short delay—the latency—that is recorded. Upon entry into the dark chamber, the door is shut closed and a foot shock is delivered. Retention of the experience is determined after various time intervals, e.g., 24 or 48 hours, by repeating the test and recording the latency. The protocol is one of many variants of the passive avoidance procedures (for review, see Rush (1988) Behav Neural Biol 50:255).

An exemplary maze testing embodiment is the water maze working memory test. In general, the method utilizes an apparatus which consists of a circular water tank. The water in the tank is made cloudy by the addition of milk powder. A clear plexiglass platform, supported by a movable stand rest on the bottom of the tank, is submerged just below the water surface. Normally, a swimming rat cannot perceive the location of the platform but it may recall it from a previous experience and training, unless it suffers from some memory impairment. The time taken to locate the platform is measured and referred to as the latency. During the experiment, all orientational cues such as ceiling lights, etc., remain unchanged. Longer latencies are generally observed with rats with some impairment to their memory.

Another memory test includes the eyeblink conditioning test, which involves the administration of white noise or steady tone that precedes a mild air puff which stimulates the subject's eyeblink.

Still another memory test which can be used is fear conditioning, e.g., either “cued” and “contextual” fear conditioning. In one embodiment, a freeze monitor administers a sequence of stimuli (sounds, shock) and then records a series of latencies measuring the recovery from shock induced freezing of the animal.

Another memory test for the lesioned animals is a holeboard test, which utilizes a rotating holeboard apparatus containing (four) open holes arranged in a 4-corner configuration in the floor of the test enclosure. A mouse is trained to poke its head into a hole and retrieve a food reward from a “baited” hole which contains a reward on every trial. There is a food reward (e.g., a Froot Loop) in every exposed hole which is made inaccessible by being placed under a screen. The screen allows the odor of the reward to emanate from the hole, but does not allow access to the reinforcer. When an individual hole is baited, a reward is placed on top of the screen, where it is accessible. The entire apparatus rests on a turntable so that it may be rotated easily to eliminate reliance on proximal (e.g., olfactory) cues. A start tube is placed in the center of the apparatus. The subject is released from the tube and allowed to explore for the baited (“correct”) hole.

As set out above, one use for the fornix-lesioned animals is for testing methylphenidate compounds for ability to enhance or inhibit memory consolidation, as well as for side effects and toxicity. In general, the subject method utilizes an animal which has been manipulated to create at least partial disruption of fornix-mediated signalling to the hippocampus, the disruption affecting memory consolidation and learned behavior in the animal. The animal is conditioned with a learning or memory regimen which results in learned behavior in the mammal in the absence of the fornix lesion. Methylphenidate compounds are administered to the animal in order to assess their effects on memory consolidation. An increase in learned behavior, relative to the absence of the test agents, indicates that the administered combination enhances memory consolidation.

In the methods of the present invention, retention of the learned behavior can be determined, for example, after at least about 12-24 hours, 14-22 hours, 16-20 hours and or 18-19 hours after completion of the learning phase to determine whether the agents promote memory consolidation. In a particular embodiment, retention of the learned behavior can be determined 24 hours after completion of the learning phase.

As used herein, a “control mammal” can be an untreated lesion mammal (i.e., a lesion animal receiving no agents or not the same combinations to be assessed), a trained control mammal (i.e., a mammal that undergoes training to demonstrate a learned behavior without any lesion) and/or an untrained control mammal (i.e., a mammal with or without a lesion, that receives no training to demonstrate a learned behavior).

C. Pharmaceutical preparations of methylphenidate compounds

In another aspect, the present invention provides pharmaceutical preparations comprising the subject methylphenidate compounds. The methylphenidate compounds for use in the subject method may be conveniently formulated for administration with a biologically acceptable, non-pyrogenic, and/or sterile medium, such as water, buffered saline, polyol (for example, glycerol, propylene glycol, liquid polyethylene glycol and the like) or suitable mixtures thereof. The optimum concentration of the active ingredient(s) in the chosen medium can be determined empirically, according to procedures well known to medicinal chemists. As used herein, “biologically acceptable medium” includes any and all solvents, dispersion media, and the like which may be appropriate for the desired route of administration of the pharmaceutical preparation. The use of such media for pharmaceutically active substances is known in the art. Except insofar as any conventional media or agent is incompatible with the activity of the methylphenidate compounds, its use in the pharmaceutical preparation of the invention is contemplated. Suitable vehicles and their formulation inclusive of other proteins are described, for example, in the book Remington's Pharmnaceutical Sciences (Remington's Pharmaceutical Sciences. Mack Publishing Company, Easton, Pa., USA 1985). These vehicles include injectable “deposit formulations”.

Pharmaceutical formulations of the present invention can also include veterinary compositions, e.g., pharmaceutical preparations of the methylphenidate compounds suitable for veterinary uses, e.g., for the treatment of live stock or domestic animals, e.g., dogs.

Methods of introduction may also be provided by rechargeable or biodegradable devices. Various slow release polymeric devices have been developed and tested in vivo in recent years for the controlled delivery of drugs. A variety of biocompatible polymers (including hydrogels), including both biodegradable and non-degradable polymers, can be used to form an implant for the sustained release of a methylphenidate compound at a particular target site. In accordance with the practice of this invention, it has been found that a dosage form and a method can be provided that administers a methylphenidate compound in a program that substantially lessens or completely compensates for tolerance in a patient. Tolerance, as defined in Pharmacology in Medicine, by Brill, p. 227 (1965) McGraw-Hill, is characterized as a decrease in effect followed by administering a drug. When tolerance develops following a single dose or a few doses over a very short time, it is referred to as acute tolerance. When the drug is administered over a more protracted period of time to show a demonstrable degree of tolerance, it is referred to as chronic tolerance. The medical literature, as exemplified in, The Pharmacological Bases of Therapeutics, by Goodman and Gilman, 8th Ed., p. 72 (1990) Pergamon Press, reported tolerance may be acquired to the effects of many drugs and this literature classifies tolerance as acute or chronic based on when it is acquired. That is, acute tolerance develops during a dosing phase of one dose or on one day, and chronic tolerance is acquired due to chronic administration typically weeks, months, and years.

In certain embodiments, particularly where the selected methylphenidate compound is one which may produce tolerance, e.g., acute tolerance, in the patient, it may desirable to formulate the compound for variable dosing, and preferably for use in a dose-escalation regimen. In preferred embodiments, the subject methylphenidate compounds are formulated to deliver a sustained and increasing dose, e.g., over at least 4 hours, and more preferably over at least 8 or even 16 hours.

In certain embodiments, representative dosage forms include hydrogel matrix containing a plurality of tiny pills. The hydrogel matrix comprises a hydrophilic polymer, such as selected from the group consisting of a polysaccharide, agar, agarose, natural gum, alkali alginate including sodium alginate, carrageenan, fucoidan, farcellaran, laminaran, hypnea, gum arabic, gum ghatti, gum karaya, gum tragacanth, locust bean gum, pectin, amylopectin, gelatin and a hydrophilic colloid. The hydrogel matrix comprises a plurality of tiny pills (such as 4 to 50), each tiny pill comprising an increasing dose population of from 100 ng ascending in dose such as 0.5 mg, 1 mg, 1.2 mg, 1.4 mg, 1.6 mg, 1.8 mg, etc. The tiny pills comprise a release rate controlling wall of 0.0 mm to 10 mm thickness to provide for the timed ascending release of drug. Representative of wall-forming materials include a triglyceryl ester selected from the group consisting of glyceryl tristearate, glyceryl monostearate, glyceryl dipalmitate, glyceryl laureate, glyceryl didecenoate and glyceryl tridecenoate. Other wall forming materials comprise polyvinyl acetate phthalate, methylcellulose phthalate, and microporous vinyl olefins. Procedures for manufacturing tiny pills are disclosed in U.S. Pat. Nos. 4,434,153; 4,721,613; 4,853,229; 2,996,431; 3,139,383 and 4,752,470, which are incorporated by reference herein.

In certain embodiments, the drug releasing beads are characterized by a dissolution profile wherein 0 to 20% of the beads undergo dissolution and release the drug in 0 to 2 hours, 20 to 40% undergo dissolution and release the drug in 2 to 4 hours, 40 to 60% exhibit dissolution and release in 4 to 6 hours, 60 to 80% in 6 to 8 hours, and 80 to 100% in 8 to 10 hours. The drug releasing beads can include a central composition or core comprising a drug and pharmaceutically acceptable composition forming ingredients including a lubricant, antioxidant, and buffer. The beads comprise increasing doses of drug, for example, 1 mg, 2 mg, 5 mg, and so forth to a high dose, in certain preferred embodiments, of 15 to 100 mg. The beads are coated with a release rate controlling polymer that can be selected utilizing the dissolution profile disclosed above. The manufacture of the beads can be adapted from, for example, Liu et al. (1994) Inter. J. of Pharm., 112:105-116; Liu et al. (1994) Inter. J. of Pharm., 112:117-124; Pharm. Sci., by Remington, 14th Ed. pp. 1626-1628 (1970); Fincher et al. (1968) J. Pharm. Sci., 57:1825-1835; and U.S. Pat. No. 4,083,949.

Another exemplary dosage form provided by the invention comprises a concentration gradient of methylphenidate compound from 1 mg to 15-600 mg coated from the former low dose to the latter high dose on a polymer substrate. The polymer can be erodible or a nonerodible polymer. The coated substrate is rolled about itself from the latter high dose at the center of the dosage form, to the former low dose at the exposed outer end of the substrate. The coated substrate is rolled from the high dose to the low dose to provide for the release of from low to high dose as the substrate unrolls or erodes. For example, 1 mg to 600 mg of methylphenidate is coated onto an erodible polymer such as an polypeptide, collagen, gelatin, or polyvinyl alcohol, and the substrate rolled concentrically from the high dose rolled over and inward to adapt a center position, and then outward towards the low dose to form an outer position. In operation, the dosage form erodes dispensing an ascending dose of methylphenidate that is released over time.

Another dosage form provided by the invention comprises a multiplicity of layers, wherein each layer is characterized by an increasing dose of drug. The phrase “multiplicity of layers” denotes 2 to 6 layers in contacting lamination. The multiplicity of layers are positioned consecutively, that is, one layer after another in order, with a first exposed layer, the sixth layer in contact with the fifth layer and its exposed surface coated with a drug impermeable polymer. The sixth layer is coated with a drug impermeable polymer to insure release of the methylphenidate compound from the first layer to the sixth layer. The first layer comprises, for example, 1 to 50 mg of drug and each successive layer comprises an additional 1 to 50 mg of drug. The biodegradable polymers undergo chemical decomposition to form soluble monomers or soluble polymer units. The biodegradation of polymers usually involves chemically or enzymatically catalyzed hydrolysis. Representative of biodegradable polymers acceptable for an increase drug loading in each layer of from 5 to 50 wt % over the first and successive layers wherein the first layer comprises 100 ng. Representative biodegradable polymers comprise a member selected from the group consisting of biodegradable poly(amides), poly(amino acids), poly(esters), poly(lactic acid), poly(glycolic acid), poly(orthoesters), poly(anhydrides), biodegradable poly(dehydropyrans), and poly(dioxinones). The polymers are known to the art in Controlled Release of Drugs, by Rosoff, Ch. 2, pp. 53-95 (1989); and in U.S. Pat. Nos. 3,811,444; 3,962,414; 4,06,747; 4,070,038; and 4,093,709.

In still other embodiments, the invention employs a dosage form comprising a polymer that releases a drug by diffusion, flux through pores, or by rupture of a polymer matrix. The drug delivery polymeric system comprises a concentration gradient, wherein the gradient is an ascent in concentration from a beginning or initial concentration to a final, or higher concentration. The dosage form comprises an exposed surface at the beginning dose and a distant nonexposed surface at the final dose. The nonexposed surface is coated with a pharmaceutically acceptable material impermeable to the passage of drug. The dosage form structure provides for a flux increase delivery of drug ascending from the beginning to the final delivered dose.

FIG. 10 illustrates such an embodiment, where the methylphenidate compound is contained within a nonabsorbable shell that releases the drug at a controlled rate.

The dosage form matrix can be made by procedures known to the polymer art. In one manufacture, 3 to 5 or more casting compositions are independently prepared wherein each casting composition comprises an increasing dose of drug with each composition overlayered from a low to the high dose. This provides a series of layers that come together to provide a unit polymer matrix with a concentration gradient. In another manufacture, the higher does is cast first followed by laminating with layers of decreasing dose to provide a polymer matrix with a drug concentration gradient. An example of providing a dosage form comprises blending a pharmaceutically acceptable carrier, like polyethylene glycol, with a known dose of a methylphenidate compound and adding it to a silastic medical grade elastomer with a cross-linking agent, like stannous octanoate, followed by casting in a mold. The step is repeated for each successive layer. The system is allowed to set, for 1 hour, to provide the dosage form. Representative polymers for manufacturing the dosage form comprise a member selected from the group consisting of olefin and vinyl polymers, condensation polymers, carbohydrate polymers, and silicon polymers as represented by poly(ethylene), poly(propylene), poly(vinyl acetate), poly(methyl acrylate), poly(isobutyl methacrylate), poly(alginate), poly(amide), and poly(silicone). The polymers and manufacturing procedures are known in Polymers, by Coleman et al., Vol. 31, pp. 1187-1230 (1990); Drug Carrier Systems, by Roerdink et al., Vol. 9, pp. 57-109 (1989); Adv. Drug Delivery Rev., by Leong et al., Vol. 1, pp. 199-233 (1987); Handbook of Common Polymers, Compiled by Roff et al., (1971) published by CRC Press; and U.S. Pat. No. 3,992,518.

In still other embodiments, the subject formulations can be a mixture of different prodrug forms of one or more different methylphenidate compounds, each prodrug form having a different hydrolysis rate, and therefore activation rate, to provide an increasing serum concentrationof the active methylphenidate compounds.

In other embodiments, the subject formulations can be a mixture different methylphenidate compounds, each compound having a different rate of adsorption (such as across the gut or epithelia) and/or serum half-life.

The dose-escalation regimen of the present invention can be used to compensate for the loss of a therapeutic effect of a methylphenidate compound, if any, by providing a method of delivery that continually compensates for the development of acute tolerance, by considering the clinical effect (E) of a drug at time (t) as a function of the drug concentration (C) according to Equation 1:

Effect= [symbol] (t, C)

In addition, the rate of drug delivered (A), in mg per hour is inversely proportional to the concentration times the clearance of the drug. As the effect varies with time and the functionality is expressed, then according to this invention (A) can be governed to ensure the therapeutic effect is maintained at a clinical value. If the effect from a drug is found clinically to decrease with time, this decline could be linear as expressed by Equation 2:

Effect₍ₜ₎=Effect₍ᵢₙᵢ₎−k_{effect}*t

wherein, Effect₍ᵢₙᵢ₎ is the clinical effect observed initially at the start of drug administration and Effect₍ₜ₎ is the effect observed at time (t) hours, k_{effect} is a proportionality constant ascertained by measuring the clinical effect (E1) at time (t1) hours and (E2) at time (t2) hours while maintaining a constant plasma concentration followed by dividing (E1) minus (E2) by (t1) minus (t2). In order to maintain a constant effect, (A) must be adjusted with the same functionality according to Equation 3:

A₍ₜ₎=A₍ᵢₙᵢ₎+k_{effect}*t

wherein A₍ᵢₙᵢ₎ is the initial drug input in mg per hour at the start of the therapy and A₍ₜ₎ is the drug input at time (t) hours, and k_{effect} is the proportionality constant presented above. If the therapeutic effect is found to decline exponentially with time, this relationship is expressed by Equation 4:

Effect₍ₜ₎=Effect₍ᵢₙᵢ₎*exp^{(-keffect*t)}

wherein Effect₍ᵢₙᵢ₎ and Effect₍ₜ₎ are as defined before, k_{effect} (or keffect) is a rate constant (h⁻¹), a unit of reciprocal hours, ascertained by measuring the clinical effect (E1) at time (t1) hours and (E2) at time (t2) hours while maintaining a constant plasma concentration followed by dividing natural log of (E1) minus natural log of (E2) by (t1) minus (t2). To maintain a constant effect, (A) must be adjusted according to Equation 5:

A₍ₜ₎=A₍ᵢₙᵢ₎*exp^{(keffect*t)}

wherein A₍ᵢₙᵢ₎ and A₍ₜ₎ are as defined before, k_{effect} is the rate constant (h⁻¹) presented above. The equations are presented in Holford et al. (1982) Pharmac. Ther., 16:143-166.

The effects defined herein refer to the pharmacological effects exhibited by methylphenidate which can be ascertained by clinical subjective observation such as described herein.

The preparations of the present invention may be given orally, parenterally, topically, or rectally. They are of course given by forms suitable for each administration route. For example, they are administered in tablets or capsule form, controlled release patch, administration by injection, infusion or inhalation; topical by lotion or ointment; and rectal by suppositories. Oral and topical administrations are preferred.

In certain preferred embodiments, the subject methylphenidate compound is delivered by way of a transdermal patch. A patch is generally a flat hollow device with a permeable membrane on one side and also some form of adhesive to maintain the patch in place on the patient's skin, with the membrane in contact with the skin so that the medication can permeate out of the patch reservoir and into and through the skin. The outer side the patch is formed of an impermeable layer of material, and the membrane side and the outer side are joined around the perimeter of the patch, forming a reservoir for the medication and carrier between the two layers.

Patch technology is based on the ability to hold an active ingredient in constant contact with the epidermis. Over substantial periods of time, methylphenidate compound, held in such a state, will eventually find its way into the bloodstream. Thus, patch technology relies on the ability of the human body to pick up drug molecules through the skin. Transdermal drug delivery using patch technology has recently been applied for delivery of nicotine, in an effort to assist smokers in quitting, the delivery of nitroglycerine to angina sufferers, the delivery of replacement hormones in post menopausal women, etc. These conventional drug delivery systems comprise a patch with an active ingredient such as a methylphenidate compound incorporated therein, the patch also including an adhesive for attachment to the skin so as to place the active ingredient in close proximity to the skin. Exemplary patch technologies are available from Ciba-Geigy Corporation and Alza Corporation. Such transdermal delivery devices can be readily adapted for use with the subject methylphenidate compounds.

The flux of methylphenidate compounds across the skin can be modulated by changing either (a) the resistance (the diffusion coefficient), or (b) the driving force (the solubility of the drug in the stratum corneum and consequently the gradient for diffusion). Various methods can be used to increase skin permeation by the subject methylphenidate compounds, including penetration enhancers, use of pro-drug versions, superfluous vehicles, iontophoresis, phonophoresis and thermophoresis. Many enhancer compositions have been developed to change one or both of these factors. See, for example, U.S. Pat. Nos. 4,006,218; 3,551,154; and 3,472,931, for example, respectively describe the use of dimethylsulfoxide (DMSO), dimethyl formamide (DMF), and N,N-dimethylacetamide (DMA) for enhancing the absorption of topically applied drugs through the stratum corneum. Combinations of enhancers consisting of diethylene glycol monoethyl or monomethyl ether with propylene glycol monolaurate and methyl laurate are disclosed in U.S. Pat. No. 4,973,468. A dual enhancer consisting of glycerol monolaurate and ethanol for the transdermal delivery of drugs is shown in U.S. Pat. No. 4,820,720. U.S. Pat. No. 5,006,342 lists numerous enhancers for transdermal drug administration consisting of fatty acid esters or fatty alcohol ethers of C2 to C4 alkanediols, where each fatty acid/alcohol portion of the ester/ether is of about 8 to 22 carbon atoms. U.S. Pat. No. 4,863,970 shows penetration-enhancing compositions for topical application comprising an active permeant contained in a penetration-enhancing vehicle containing specified amounts of one or more cell-envelope disordering compounds such as oleic acid, oleyl alcohol, and glycerol esters of oleic acid; a C2 or C3 alkanol; and an inert diluent such as water. Other examples are included in the teachings of U.S. Pat. No. 4,933,184 which discloses the use of menthol as a penetration enhancer; U.S. Pat. No. ,229,130 discloses the use of vegetable oil (soybean and/or coconut oil) as a penetration enhancer; and U.S. Pat. No. 4,440,777 discloses the use of eucalyptol as a penetration enhancer.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrastemal injection and infusion.

The phrases “systemic administration,” “administered systemically,” “peripheral administration” and “administered peripherally” as used herein mean the administration of a compound, drug or other material other than directly into the central nervous system, such that it enters the patient's system and, thus, is subject to metabolism and other like processes, for example, subcutaneous administration.

These compounds may be administered to humans and other animals for therapy by any suitable route of administration, including orally, nasally, as by, for example, a spray, rectally, intravaginally, parenterally, intracistemally and topically, as by powders, ointments or drops, including buccally and sublingually.

Regardless of the route of administration selected, the compounds of the present invention, which may be used in a suitable hydrated form, and/or the pharmaceutical compositions of the present invention, are formulated into pharmaceutically acceptable dosage forms such as described below or by other conventional methods known to those of skill in the art.

Actual dosage levels of the active ingredients in the pharmaceutical compositions of this invention may be varied so as to obtain an amount of the active ingredient which is effective to achieve the desired therapeutic response for a particular patient, composition, and mode of administration, without being toxic to the patient.

The selected dosage level will depend upon a variety of factors including the activity of the particular compound of the present invention employed, or the ester, salt or amide thereof, the route of administration, the time of administration, the rate of excretion of the particular compound being employed, the duration of the treatment, other drugs, compounds and/or materials used in combination with the particular methylphenidate compounds employed, the age, sex, weight, condition, general health and prior medical history of the patient being treated, and like factors well known in the medical arts.

A physician or veterinarian having ordinary skill in the art can readily determine and prescribe the effective amount of the pharmaceutical composition required. For example, the physician or veterinarian could start doses of the compounds of the invention employed in the pharmaceutical composition at levels lower than that required in order to achieve the desired therapeutic effect and gradually increase the dosage until the desired effect is achieved.

In general, a suitable daily dose of a compound of the invention will be that amount of the compound which is the lowest dose effective to produce a therapeutic effect. Such an effective dose will generally depend upon the factors described above. Generally, intravenous, intracerebroventricular and subcutaneous doses of the compounds of this invention for a patient will range from about 0.0001 to about 100 mg per kilogram of body weight per day.

If desired, the effective daily dose of the active compound may be administered as two, three, four, five, six or more sub-doses administered separately at appropriate intervals throughout the day, optionally, in unit dosage forms.

The term “treatment” is intended to encompass also prophylaxis, therapy and cure.

The patient receiving this treatment is any animal in need, including primates, in particular humans, and other mammals such as equines, cattle, swine and sheep; and poultry and pets in general.

The compound of the invention can be administered as such or in admixtures with pharmaceutically acceptable carriers and can also be administered in conjunction with other psychoactive drugs such as stimulants, antidepressants, modulators of neurotransmitters, and anticonvulsants. Conjunctive therapy thus includes sequential, simultaneous and separate administration of the active compound in a way that the therapeutic effects of the first administered one is not entirely disappeared when the subsequent is administered.

While it is possible for a compound of the present invention to be administered alone, it is preferable to administer the compound as a pharmaceutical formulation (composition). The methylphenidate compounds according to the invention may be formulated for administration in any convenient way for use in human or veterinary medicine.

Thus, another aspect of the present invention provides pharmaceutically acceptable compositions comprising a therapeutically effective amount of one or more of the compounds described above, formulated together with one or more pharmaceutically acceptable carriers (additives) and/or diluents. As described in detail below, the pharmaceutical compositions of the present invention may be specially formulated for administration in solid or liquid form, including those adapted for the following: (1) oral administration, for example, drenches (aqueous or non-aqueous solutions or suspensions), tablets, boluses, powders, granules, pastes for application to the tongue; (2) parenteral administration, for example, by subcutaneous, intramuscular or intravenous injection as, for example, a sterile solution or suspension; (3) topical application, for example, as a cream, ointment or spray applied to the skin; or (4) intravaginally or intrarectally, for example, as a pessary, cream or foam. However, in certain embodiments the subject compounds may be simply dissolved or suspended in sterile water.

The phrase “pharmaceutically acceptable carrier” as used herein means a pharmaceutically acceptable material, composition or vehicle, such as a liquid or solid filter, diluent, excipient, solvent or encapsulating material, involved in carrying or transporting the subject regulators from one organ, or portion of the body, to another organ, or portion of the body. Each carrier must be “acceptable” in the sense of being compatible with the other ingredients of the formulation and not injurious to the patient. Some examples of materials which can serve as pharmaceutically acceptable carriers include (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

As set out above, certain embodiments of the present methylphenidate compounds may contain a basic functional group, such as amino or alkylamino, and are, thus, capable of forming pharmaceutically acceptable salts with pharmaceutically acceptable acids. The term “pharmaceutically acceptable salts” in this respect, refers to the relatively non-toxic, inorganic and organic acid addition salts of compounds of the present invention. These salts can be prepared in situ during the final isolation and purification of the compounds of the invention, or by separately reacting a purified compound of the invention in its free base form with a suitable organic or inorganic acid, and isolating the salt thus formed. Representative salts include but are not limited to following: 2-hydroxyethanesulfonate, 2-naphthalenesulfonate, 3-hydroxy-2-naphthoate, 3-phenylpropionate,acetate, adipate, alginate, amsonate, aspartate, benzenesulfonate, benzoate, besylate, bicarbonate, bisulfate, bitartrate, borate, butyrate, calcium edetate, camphorate, camphorsulfonate, camsylate, carbonate, citrate, clavulariate, cyclopentanepropionate, digluconate, dodecylsulfate, edetate, edisylate, estolate, esylate, ethanesulfonate, fumarate, gluceptate, glucoheptanoate, gluconate, glutamate, glycerophosphate, glycollylarsanilate, hemisulfate, heptanoate, hexafluorophosphate, hexanoate, hexylresorcinate, hydrabamine, hydrobromide, hydrochloride, hydroiodide, hydroxynaphthoate, iodide, isothionate, lactate, lactobionate, laurate, laurylsulphonate, malate, maleate, mandelate, mesylate, methanesulfonate, methylbromide, methylnitrate, methylsulfate, mucate, naphthylate, napsylate, nicotinate, nitrate, N-methylglucamine ammonium salt, oleate, oxalate, palmitate, pamoate, pantothenate, pectinate, persulfate, phosphate, phosphate/diphosphate, picrate, pivalate, polygalacturonate, propionate, p-toluenesulfonate, salicylate, stearate, subacetate, succinate, sulfate, sulfosaliculate, suramate, tannate, tartrate, teoclate, thiocyanate, tosylate, triethiodide, undecanoate, and valerate salts, and the like. (See, for example, Berge et al. (1977) “Pharmaceutical Salts”, J. Pharm. Sci. 66:1-19)

In certain embodiments, the pharmaceutically acceptable salts of the subject compounds include the conventional non-toxic salts of the compounds, e.g., from non-toxic organic or inorganic acids. Particularly suitable are salts of weak acids. For example, such conventional non-toxic salts include those derived from inorganic acids such as hydrochloride, hydrobromic, hydriodic, cinnamic, gluconic, sulfuiric, sulfamic, phosphoric, nitric, and the like; and the salts prepared from organic acids such as acetic, propionic, succinic, glycolic, stearic, lactic, maleic, tartaric, citric, ascorbic, palmitic, maleic, hydroxymaleic, phenylacetic, glutamic, benzoic, salicyclic, sulfanilic, 2-acetoxybenzoic, fumaric, toluenesulfonic, methanesulfonic, ethane disulfonic, oxalic, isothionic, and the like.

In other cases, the compounds of the present invention may contain one or more acidic functional groups and, thus, are capable of forming pharmaceutically acceptable salts with pharmaceutically acceptable bases. The term “pharmaceutically acceptable salts” in these instances refers to the relatively non-toxic, inorganic and organic base addition salts of compounds of the present invention. These salts can likewise be prepared in situ during the final isolation and purification of the compounds, or by separately reacting the purified compound in its free acid form with a suitable base, such as the hydroxide, carbonate or bicarbonate of a pharmaceutically acceptable metal cation, with ammonia, or with a pharmaceutically acceptable organic primary, secondary or tertiary amine. Representative alkali or alkaline earth salts include the lithium, sodium, potassium, calcium, magnesium, and aluminum salts and the like. Representative organic amines useful for the formation of base addition salts include ethylamine, diethylamine, ethylenediamine, ethanolamine, diethanolamine, piperazine and the like. (See, for example, Berge et al., supra) Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Formulations of the present invention include those suitable for oral, nasal, topical (including buccal and sublingual), rectal, vaginal and/or parenteral administration. The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient which can be combined with a carrier material to produce a single dosage form will generally be that amount of the compound which produces a therapeutic effect. Generally, out of one hundred per cent, this amount will range from about 1 per cent to about ninety-nine percent of active ingredient, preferably from about 5 per cent to about 70 per cent, most preferably from about 10 per cent to about 30 per cent.

Methods of preparing these formulations or compositions include the step of bringing into association a compound of the present invention with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

Formulations of the invention suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present invention as an active ingredient. A compound of the present invention may also be administered as a bolus, electuary or paste.

In solid dosage forms of the invention for oral administration (capsules, tablets, pills, dragees, powders, granules and the like), the active ingredient is mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, cetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents. In the case of capsules, tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions of the present invention, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions which can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions which can be used include polymeric substances and waxes. The active ingredient can also be in micro-encapsulated form, if appropriate, with one or more of the above-described excipients.

Liquid dosage forms for oral administration of the compounds of the invention include pharmaceutically acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations of the pharmaceutical compositions of the invention for rectal or vaginal administration may be presented as a suppository, which may be prepared by mixing one or more compounds of the invention with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active methylphenidate compound.

Formulations of the present invention which are suitable for vaginal administration also include pessaries, tampons, creams, gels, pastes, foams or spray formulations containing such carriers as are known in the art to be appropriate.

Dosage forms for the topical or transdermal administration of a compound of this invention include powders, sprays, ointments, pastes, creams, lotions, gels, solutions, patches and inhalants. The active compound may be mixed under sterile conditions with a pharmaceutically acceptable carrier, and with any preservatives, buffers, or propellants which may be required.

The ointments, pastes, creams and gels may contain, in addition to an active compound of this invention, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to a compound of this invention, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

In certain preferred embodiments, the subject compound(s) are formulated as part of a transdermal patch. Transdermal patches have the added advantage of providing controlled delivery of a compound of the present invention to the body. Such dosage forms can be made by dissolving or dispersing the methylphenidate compounds in the proper medium. Absorption enhancers can also be used to increase the flux of the methylphenidate compounds across the skin. The rate of such flux can be controlled by either providing a rate-controlling membrane or dispersing the compound in a polymer matrix or gel.

The “free base form” of methylphenidate relates to a form in which methylphenidate can be incorporated into the patch. It will be appreciated that the methylphenidate may be complexed, for example, with elements of the drug-retaining matrix of the patch and, as such, the methylphenidate may not necessarily be in the form of the free base, when actually retained by the patch.

The patch preferably comprises a drug-impermeable backing layer. Suitable examples of drug-impermeable backing layers which may be used for transdermal or medicated patches include films or sheets of polyolefins, polyesters, polyurethanes, polyvinyl alcohols, polyvinyl chlorides, polyvinylidene chloride, polyamides, ethylene-vinyl acetate copolymer (EVA), ethylene-ethylacrylate copolymer (EEA), vinyl acetate-vinyl chloride copolymer, cellulose acetate, ethyl cellulose, metal vapour deposited films or sheets thereof, rubber sheets or films, expanded synthetic resin sheets or films, non-woven fabrics, fabrics, knitted fabrics, paper and foils. Preferred drug-impermeable, elastic backing materials are selected from polyethylene tereplithalate (PET), polyurethane, ethylene-vinyl acetate copolymer (EVA), plasticized polyvinylchloride, woven and non-woven fabric. Especially preferred is non-woven polyethylenetereplithalate (PET). Other backings will be readily apparent to those skilled in the art.

The term ‘block copolymer’, in the preferred adhesives of the invention, refers to a macromolecule comprised of two or more chemically dissimilar polymer structures, terminally connected together (Block Copolymers: Overview and Critical Survey, Noshay and McGrath, 1977). These dissimilar polymer structures, sections or segments, represent the ‘blocks’ of the block copolymer. The blocks may generally be arranged in an A-B structure, an A-B-A structure, or a multi-block -(A-B)n- system, wherein A and B are the chemically distinct polymer segments of the block copolymer.

It is generally preferred that the block copolymer is of an A-B-A structure, especially wherein one of A and B is an acrylic-type polymeric unit. It will be appreciated that the present invention is also applicable using block copolymers which possess three or more different blocks, such as an A-B-C block copolymer. However, for convenience, reference hereinafter to block copolymers will assume that there are only A and B sub-units, but it will be appreciated that such reference also encompasses block copolymers having more than two different sub-units, unless otherwise specified.

It will be appreciated that the properties of block copolymers are very largely determined by the nature of the A and B blocks. Block copolymers commonly possess both ‘hard’ and ‘soft’ segments. A ‘hard’ segment is a polymer that has a glass transition temperature (Tg) and/or a melting temperature (Tm) that is above room temperature, while a ‘soft’ segment is a polymer that has a Tg (and possibly a Tm) below room temperature. The different segments are thought to impart different properties to the block copolymer. Without being constrained by theory, it is thought that association of the hard segments of separate block copolymer units result in physical cross-links within the block copolymer, thereby promoting cohesive properties of the block copolymer. It is particularly prefer-red that the hard segments of the block copolymers form such physical close associations.

The block copolymers useful in the present invention preferably are acrylic block copolymers. In acrylic block copolymers, at least one of the blocks of the block copolymer is an acrylic acid polymer, or a polymer of an acrylic acid derivative. The polymer may be composed of just one repeated monomer species. However, it will be appreciated that a mixture of monomeric species may be used to form each of the blocks, so that a block may, in itself, be a copolymer. The use of a combination of different monomers can affect various properties of the resulting block copolymer. In particular, variation in the ratio or nature of the monomers used allows properties such as adhesion, tack and cohesion to be modulated, so that it is generally advantageous for the soft segments of the block copolymer to be composed of more than one monomer species.

It is preferred that alkyl acrylates and alkyl methacrylates are polymerized to form the soft portion of the block copolymer. Alkyl acrylates and alkyl methacrylates are thought to provide properties of tack and adhesion. Suitable alkyl acrylates and alkyl methacrylates include n-butyl acrylate, n-butyl methacrylate, hexyl acrylate, 2-ethylbutyl acrylate, isooctyl acrylate, 2-ethylhexyl acrylate, 2-ethylhexyl methacrylate, decyl acrylate, decyl methacrylate, dodecyl acrylate, dodecyl methacrylate, tridecylacrylate and tridecyl methacrylate, although other suitable acrylates and methacrylates will be readily apparent to those skilled in the art. It is preferred that the acrylic block copolymer comprises at least 50% by weight of alkyl acrylate or alkyl methacrylate(co)polymer.

Variation in the components of the soft segment affects the overall properties of the block copolymer, although the essential feature remains the cross-inking of the soft segments. For example, soft segments essentially consisting of diacetone acrylamide with either butyl acrylate and/or 2-ethylhexyl acrylate, in approximately equal proportions, work well, and a ratio by weight of about 3:4:4 provides good results. It is preferred that diacetone acrylamide, or other polar monomer, such as hydroxyethylmethacrylate or vinyl acetate, be present in no more than 50% w/w of the monomeric mix of the soft segment, as this can lead to reduced adhesion, for example. The acrylate component may generally be varied more freely, with good results observed with both 2-ethylhexyl acrylate and butyl acrylate together or individually.

As noted above, ratios of the various monomers are generally preferred to be approximately equal. For adhesives, this is preferred to be with a polar component of 50% or less of the soft segment, with the apolar portion forming up to about 85% w/w, but preferably between about 50 and 70% w/w. In the example above, this is about 72% (4+4) a polar to about 18% (3) polar.

In general, it is particularly preferred that any apolar monomer used does not confer acidity on the adhesive. Adhesives of the invention are preferably essentially neutral, and this avoids any unnecessary degeneration of the methylphenidate.

Limiting active functionalities, especially those with active hydrogen, is generally preferred, in order to permit wide use of any given formulation of adhesive without having to take into account how it is likely to interact, chemically, with its environment. Thus, a generally chemically inert adhesive is preferred, in the absence of requirements to the contrary.

As discussed above, polymers suitable for use as the hard portion of the block copolymer possess glass transition temperatures above room temperature. Suitable monomers for use in forming the hard segment polymer include styrene,(x-methylstyrene, methyl methacrylate and vinyl pyrrolidone, although other suitable monomers will be readily apparent to those skilled in the art. Styrene and polymethylmethacrylate have been found to be suitable for use in the formation of the hard segment of the block copolymers. It is preferred that the hard portion of the block copolymer forms from 3-30% w/w of the total block copolymer, particularly preferably from 5-15% w/w.

The block copolymer is further characterized in that the soft portions contain a degree of chemical cross-linking. Such cross-linking may be effected by any suitable cross-linking agent. It is particularly preferable that the cross-linking agent be in the form of a monomer suitable for incorporation into the soft segment during polymerization. Preferably the cross-linking agent has two, or more, radically polymerizable groups, such as a vinyl group, per molecule of the monomer, at least one tending to remain unchanged during the initial polymerization, thereby to permit cross-linking of the resulting block copolymer.

Suitable cross-linking agents for use in the present invention include divinylbenzene, methylene bis-acrylamide, ethylene glycol di(meth)acrylate, ethyleneglycol tetra(meth)acrylate, propylene glycol di(meth)acrylate, butylene glycoldi(meth)acrylate, or trimethylolpropane tri(meth)acrylate, although other suitable cross-linking agents will be readily apparent to those skilled in the art. A preferred cross-linking agent is tetraethylene glycol dimethacrylate. It is preferred that the cross-linking agent comprises about 0.01-0.6% by weight of the block copolymer, with 0.1-0.4% by weight being particularly preferred.

Methods for the production of block copolymers from their monomeric constituents are well known. The block copolymer portions of the present invention may be produced by any suitable method, such as step growth, anionic, cationic and free radical methods (Block Copolymers, supra). Free radical methods are generally preferred over other methods, such as anionic polymerization, as the solvent and the monomer do not have to be purified.

Suitable initiators for polymerization include polymeric peroxides with more than one peroxide moiety per molecule. An appropriate choice of reaction conditions is well within the skill of one in the art, once a suitable initiator has been chosen.

The initiator is preferably used in an amount of 0.005-0.1% by weight of the block copolymer, with 0.01-0.05% by weight being particularly preferred, although it will be appreciated that the amount chosen is, again, well within the skill of one in the art. In particular, it is preferred that the amount should not be so much as to cause instant gelling of the mix, nor so low as to slow down polymerization and to leave excess residual monomers. A preferred level of residual monomers is below 2000 ppm.

It will also be appreciated that the amount of initiator will vary substantially, depending on such considerations as the initiator itself and the nature of the monomers.

The block copolymers are adhesives, and preferably are pressure sensitive adhesives. Pressure sensitive adhesives can be applied to a surface by hand pressure and require no activation by heat, water or solvent. As such, they are particularly suitable for use in accordance with the present invention.

The block copolymers may be used without tackifiers and, as such, are particularly advantageous. However, it will be appreciated that the block copolymers may also be used in combination with a tackifier, to provide improved tack, should one be required or desired. Suitable tackifiers are well known and will be readily apparent to those skilled in the art.

Without being constrained by theory, it is thought that the combination of chemical cross-links between the soft segments of the copolymer combined with the, generally, hydrophobic interaction, or physical cross-linking, between the hard portions results in a “matrix-like” structure. Copolymers having only physical cross-linking of the hard segments are less able to form such a matrix. It is believed that the combination of both forms of cross-linking of the block copolymers provides good internal strength (cohesion) and also high drug storage capacity.

More particularly, it is believed that the hard segments associate to form “islands”, or nodes, with the soft segments radiating from and between these nodes.

There is a defined physical structure in the “sea” between the islands, where the soft segments are cross-linked, so that there is no necessity for extensive intermingling of the soft segments. This results in a greater cohesion of the whole block copolymer while, at the same time, allowing shortened soft segment length and still having as great, or greater, distances between the islands, thereby permitting good drug storage capacity.

The block copolymer preferably cross-links as the solvent is removed, so that cross-linking can be timed to occur after coating, this being the preferred method.

Accordingly, not only can the block copolymer easily be coated onto a surface, but the complete solution can also be stored for a period before coating. Accordingly, in the manufacturing process of the patches, the process preferably comprises polymerizing the monomeric constituents of each soft segment in solution, then adding the constituents of the hard segment to each resulting solution and polymerizing the resulting mix, followed by cross-linking by removal of any solvent or solvent system, such as by evaporation. If the solution is to be stored for any length of time, it may be necessary to keep the polymer from precipitating out, and this may be achieved by known means, such as by suspending agents or shaking. It may also be necessary to select the type of polymers that will be subject to substantially no cross-linking until the solvent is evaporated.

In general, it is preferred that the adhesive possesses a minimum number of functionalities having active hydrogen, in order to avoid undesirable reactions/interactions, such as with any drug that it is desired to incorporate into the adhesive material. It will be appreciated that this is only a preferred restriction, and that any adhesive may be tailored by one skilled in the art to suit individual requirements.

Suitable monomers for use in forming the hard segment include styrene, a-methylstyrene, methyl methacrylate and vinyl pyrrolidone, with the preferred proportion of the hard segment being between 5 and 15 percent w/w. In particular, it is advantageous to use the compounds of WO 99/02141, as it is possible to load over 30 percent of drug into such a system.

Thus, in the patches of the present invention, it is generally possible to calculate the amount of drug required and determine the appropriate patch size with a given drug loading in accordance with a patient's body weight, and this can be readily calculated by those skilled in the art.

In certain embodiments, small amounts of plasticizer, such as isopropyl myristate (IPM), are incorporated. This has the advantage of helping to solubilize the methylphenidate as well as rendering the adhesive less rough on the skin. Levels of between 2 and 25%, by weight, are generally useful, with levels of between 3 and 20% being more preferred and levels of 5 to 15% especially about 10%, being most preferred. Other plasticizers may also be used, and susitable plasticizers will be readily apparent to those skilled in the art. In particular, in this embodiment, it is preferred to employ the adhesives of WO 99/02141. It has been found that levels of about 30% methylphenidate are stable in the patches of the invention, with preferred levels being between 15 and 25%, preferably 20%.

Plasticizers generally take the form of oily substances introduced into the adhesive polymer. The effect of the introduction of such oily substances is to soften the physical structure of the adhesive whilst, at the same time, acting at the interface between the adhesive and the skin, thereby helping to somewhat weaken the adhesive, and to reduce exfoliation.

The free base oil may be obtained by basifying methylphenidate hydrochloride, or any other suitable salt, with a suitable base, in the presence of a hydrophilic solvent, especially water, and organic solvent. For instance, water and ethyl acetate, in approximately equal proportions, work well, with ammonia serving as the basifying agent. The water may then be removed and the preparation washed with further water, or other aqueous preparation, after which the preparation may be suitably extracted with ether, for example, after having removed the ethyl acetate. It is preferred to keep the preparation under an inert atmosphere, especially after completion.

Whilst it will be appreciated that patches of the present invention may be removed from the patient at any time, once it is desired to terminate a given dose, this can have the disadvantage of providing an opportunity for potential drug abuse of the partially discharged patch. Abuse of methylphenidate is highly undesirable.

In certain embodiments, it may be advantage to use a patch tailored to have delivered the majority of the methylphenidate that it is capable of delivering, in a 24 hour period, by about 8 hours after application, so that a patch can be left in place, and levels of drug still diminish appreciably. It is advantageous that the drug delivery profile has first order kinetics, so that the majority of the drug is delivered during the main part of the day and, even if the patient omits to remove the patch, the drug is moving towards exhaustion by the end of the day, and the amount of drug is dropping rapidly.

It will be appreciated that patches of the invention may be constructed in any suitable manner known in the art of the manufacture of transdermal patches. The patches may simply comprise adhesive, drug and backing, or may be more complex, such as having edging to prevent seepage of drug out of the sides of the patch. Patches may also be multi-layered, for example.

Ophthalmic formulations, eye ointments, powders, solutions and the like, are also contemplated as being within the scope of this invention.

Pharmaceutical compositions of this invention suitable for parenteral administration comprise one or more compounds of the invention in combination with one or more pharmaceutically acceptable sterile isotonic aqueous or nonaqueous solutions, dispersions, suspensions or emulsions, or sterile powders which may be reconstituted into sterile injectable soultions or dispersions just prior to use, which may contain antioxidants, buffers, bacteiostats, soutes which render the formulation isotonic with the blood of the intended recipient or suspending or thickening agents.

Examples of suitable aqueous and non aqueous carriers which may be employed in the pharmaceutical compositions of the invention include water, enthanol, polyols (such as glyccrol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol,phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monosterate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution which, in turn, may depend upon crystal size and crystalline form. Alternatively, delayed absorption of a parentally administered drug form is acccomplished by dissolving or suspending the drug in an oil vehicle.

injectable depot forms are made by forming microencapsule matrices of the subject compounds in biodegradable polymers such as polylactide-polyglycolide. Depending on the ration of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissue.

When the compounds of the present invention are administered as pharmaceuitcals, to humans and animals, they cna be given per se or as a pharmaceutical composition containing, for example, 0.1 to 99.5% (more preferably, 0.5 to 90%) of active ingredient in combination with a pharmaceutically acceptable carrier.

The addition of the active compound of the invention to animal feed is preferably accomplished by preparting an appropriate feed premix containing the active compound in an effective amount and incorporating the premix into the complete ration.

Alternatively, an intermediate concentrate or feed supplement containing the active compound in an effective amount and incorporating the premix into the complete ration.

Alternatively, an intermediate concentrate or feed supplement containing the active compound in an effective amound and incorporating the premix into the complete ration.

Alternatively, an intermediate concentrate or feed supplement containing the active ingreedient can be blended into the feed. The way in which such feed premixes and complete rations can be prepared and administered are described in reference books (such as “Applied Animal Nutrition”, W. H. Freedman and Co., San Francisco, U.S.A., 1969 or “Livestock Feeds and Feeding” O and B books, Corvallis, Oreg., U.S.A., 1977).

IV. Exemplary Uses of the Compounds of the Invention

In various embodiments, the present invention contemplates modes of treatment and prophylaxis which utilize one or more of the subject methylphenidate compounds. Those agents may be useful for altering (increasing or decreasing) the occurrence of learning and/or memory capacity of the organism. In other embodiments, the preparations of the present invention can be used simply to enhance normal memory function.

In various other embodiments, the present invention contemplates modes of treatment and prophylaxis which utilize one or more of the subject methylphenidate compounds to alter defects in attention span and/or focus in an organism. The enhancement and/or restoration of attention span in an organism has positive behavioral, social, and psychological consequences. Additionally, enhancement of attention span can improve memory and learning.

In certain embodiments, the subject method can be used to treat patients who have been diagnosed as having or at risk of developing disorders in which diminished declarative memory is a symptom, e.g., as opposed to procedural memory. The subject method can also be used to treat normal individuals for whom improved declarative memory is desired.

Memory disorders which can be treated according to the present invention may have a number of origins: a functional mechanism (anxiety, depression), physiological aging (age-associated memory impairment, mild cognitive impairment, etc.), drugs, or anatomical lesions (dementia). Indications for which such preparations may be useful include learning disabilities, memory impairment, e.g., due to toxicant exposure, brain injury, age, schizophrenia, epilepsy, mental retardation in children, Down's Syndrome and senile dementia, including Alzheimer's diease. It can be used to treat Anterior Communicating Artery Syndrome and other Stroke syndromes. The subject method can also be used to treat (lessen the severity of) or as a prophylaxis against memory impairment as a consequence to ischemia or hypoxia, such as may the consequence of reduced blood flow or blood volume (including heart bypass surgery or dieases involving reduced or impaired cardiac output) or exposure to low oxygen conditions.

Although in certain embodiments, attention deficit disorder (ADD), attention deficit hyperactivity disorder (ADHD), and AIDS-related dementia may respond to treatment with a subject compound, in certain embodiments, the patient's memory loss is not associated with one of these conditions.

PDDs are a class of disorders defined by both American and International diagnostic systems (i.e., the Diagnostic and Statistical Manual of Mental Disorders, 4th edition (DSM-IV) and World Health Organization: International Classification of Dieases, Tenth revision (ICD-10)). The spectrum of PDDs include disorders such as Autism, Aspergers, ADD, and ADHD. PDDs are typically characterized by multiple distortions in the development of basic psychological functions that are involved in the development of social skills and language, such as attention, perception, reality testing and motor movement.

A subject which displays signs of developmentally inappropriate inattention, impulsivity and hyperactivity is typically diagnosed as having ADD and/or ADHD. With these disorders, there can be marked disturbances of organization, distractibility, impulsivity, restlessness, and other disturbances of language and/or social behavior. A combination of psychiatric care and medicine is typically used for treating individuals with ADD and ADHD. Psychiatric care may include, for example: individual psychotherapy therapy, group therapy, behavior modification, and art therapy. A skilled mental health practioner (e.g., a psychiatrist, psychologist, therapist, social worker) can make a diagnosis of ADD or ADHD based on observations of the patient in view of the criteria set forth in the DSM-IV. The skilled practioner has a variety of diagnostic measures available to assess attention deficit related disorders, as well as a variety of measures for assessing improvements in attention span or the course of treatment.

An attention-deficit disorder (ADD) is a developmental disorder characterized by developmentally inappropriated degrees of inattention, overactivity, and impulsivity. Symptoms are neurologically-based, arise in early childhood, and are chronic in nature in most cases. Symptoms are not due to gross neurological impairment, sensory impairment, language or motor impairment, mental retardation, or emotional disturbance. Attention Deficit Disorders are the most common psychiatric disorders in children with reported rates ranging from 4% to 9%. Attention Deficit Disorder (ADD) is characterized by inattention and impulsivity and may be present with hyperactivity (ADHD) (Shaywitz et al. 1984). Other characteristics may include aggressiveness, stealing, lying, truancy, setting fires, running away, explosiveness, cognitive and learning problems as well as poor social skills (Campbell et al. 1992). It is four to five times more frequent in boys than girls.

ADD with and without hyperactivity are separate and unique childhood and adult disorders. They are not subtypes of an identical attention disturbance. It has been noted that children with ADD/−H are more frequently described as depressed, learning disabled, or “lazy” while children with ADD/+H are more frequently labeled as conduct or behavior disordered.

Children with ADD or ADHD, the most common of the psychiatric disorders that appear in childhood, are often the subject of great concern on the part of parents and teachers. These children are unable to stay focused on a task, cannot sit still, act without thinking, and rarely finish anything. If untreated, the disorder can have long-term effects on a child's ability to make friends or do well at school or in other activities. Over time, children with ADD or ADHD may develop depression, lack of self-esteem, and other emotional problems.

Experts estimate that attention deficit related disorders affect 3-5% percent of school-age children and two to three as many boys as girls. Let untreated, children with such disorders have higher than normal rates of injury. ADHD frequently co-occurs with other problems, such as depression and anxiety disorders, conduct disorder, drug abuse, or antisocial behavior.

Characteristics of attention deficit disorders have been demonstrated to arise in early childhood for most individuals. This disorder is marked by chronic behaviors lasting at least six months with an onset often before seven years of age. At this time, four subtypes of ADHD have been defined. These include the following:

1. ADHD—Inattentive type

2. ADHD—hyperactive/impulsive type

3. ADHD—combined type

4. ADHD—not otherwise specified is defined by an individual who demonstrates some characteristics but an insufficient number of symptoms to reach a full diagnosis. These symptoms, however, disrupt everyday life.

The American Psychiatric Association Diagnostic and Statistical Manual (DSM-IV) criteria for diagnosing attention deficit disorders include:

A. Either (1) or (2)

(1). six or more of the following symptoms of inattention have persisted for at least 6 months to a degree that is maladaptive and inconsistent with developmental level.

Inattention: People who are inattentive have a hard time keeping their mind on any one thing and may get bored with a task afte only a few minutes. They may give effortless, automatic attention ot activities and things they enjoy. But focusing delibeate, conscious attention to organizing and completing a task or learning something new is difficult.

(a) often fails to give close attention to details or makes careless mistakes in schoolwork, work, or other activities

(b) often has difficulty sustaining attention in tasks or play activities

(c) often does not seem to lesten when spoken to directly

(d) often does not follow through on instructions and fails to finish schoolwork, chores, or duties in the workplace (not due to oppositional behavior or failure to understand instructions)

(e) often has difficulty organizing tasks and activities

(f) often avoids, dislikes, or is reluctant to engage in tasks that require sustained mental effort (such as schoolwork or homework).

(g) often loses things necessary for tasks or activities (e.g. toys, school assignments, pencils, books, or tools)

(h) is often easily distracted by extranous stimuli

(i) is often forgetful in daily activities

(2). six (or more) of the following symptoms of hyperactivity-impulsivity have persisted for at least 6 months to a degree that is maladaptive and inconsistent with developmental level

Hyperactivity: People who are hyperactive always seem to be in motion. They can't sit still. They may dash around or talk incessantly. Sitting still through a lesson can be an impossible task. Hyperactive children may squirm in their seat, roam around the room, touch everything in reach, or noisily tap their pencil. Hyperactive teens and adults may feel intensely restless. They may be fidgety or, they may try to do several things at once, bounding around from one activity to the next.

(a) often fidgets with hands or feet or squirms in seat

(b) often leaves seat in classroom or in other situations in which remaining seated is expected

(c) often runs about or climbs excessively in situations in which it is inappropriate (in adolescents or adults, may be limited to subjective feelings of restlessness)

(d) often has difficulty playing or engaging in leisure activities quietly

(e) is often “on the go” or often acts as if “driven by a motor”

(f) often talks excessively

Impulsivity: People who are overly impulsive seem unable to curb their immediate reactions or think before they act. As a result, they may blurt out inappropriate comments. Their impulsivity may make it hard for them to wait for things they want or to take their turn in games. Their inability to wait appropriately may lead to frustration and violence.

(g) often blurts out answers before questions have been completed

(h) often has difficulty awaiting turn

(i) often interrupts or intrudes on others (e.g. butts into conversations or games)

B. Some hyperactive-impulsive or inattentive symptoms that caused impairment were present before age 7 years.

C. Some impairment from the symptoms is present in two or more settings (e.g. at school [or work] and at home).

D. There must be clear evidence of clinically significant impairment in social, academic, or occupational functioning. E. The symptoms do not occur exclusively during the course of a Pervasive Developmental Disorder, Schizophrenia, or other Psychotic Disorder and are not better accounted for by another mental disorder (e.g. Mood Disorder, Anxiety Disorder, Dissociative Disorder, or a Personality Disorder)

More seriously, nearly half of all individuals with attention deficit disorders—mostly boys—tend to have another condition, called oppositional defiant disorder. These individuals may overreact or lash out when they feel badly about themselves. They may be stubborn, have outbursts of temper, or act belligerent or defiant. Sometimes this progresses to more serious conduct disorders. Individuals with this combination of problems are at risk of getting in trouble at school, and even with the police. They may take unsafe risks and break laws—they may steal, set fires, destroy property, and drive recklessly.

At some point, many individuals with attention deficit disorders—mostly younger children and boys—experience other emotional disorders. About one-fourth feel anxious. They feel tremendous worry, tension, or uneasiness, even when there's nothing to fear. Because the feelings are scarier, stronger, and more frequent than normal fears, they can affect the person's thinking and behavior. Others experience depression. Depression goes deyond ordinary sadness—may feel so “down” that they feel hopeless and unable to deal with everyday tasks. Depression can disrupt sleep, appetite, and the ability to think.

Attention deficit disorders have a tremendous impact on individuals. The lack of attention span, as well as the accompany restlessness, hyperactivity, and impulsivity, have significant social, behavioral, and psychological impacts. Furthermore, deficits in attention span, as well as the accompanying hyperactivity and restlessness can greatly impact memory and learning. Improvements in attention span and/or a decrease in the restlessness and impulsivity of individuals with an attention deficit disorder would greatly impact these individuals.

As outlined above, criteria for the diagnosis of an attention deficit disorder are detailed in the DSM, and a skilled practitioner can readily observe and test subjects to arrive at a diagnosis. Examples of practitioners equipped to make a diagnosis include psychiatrists, psychologists, physicians, neurologist, therapists, and social workers.

While the art teaches the use of racemic methylphenidate (Ritalin) for the treatment of ADHD, it does not, to the knowledge of the inventors, recognize that l-threo-methylphenidate may be effective in treating attention deficit disorders.

In certain embodiments, the invention contemplates the treatment of amnesia. Amnesias are described as specific defects in declarative memory. Faithful encoding of memory requires a registration, rehearsal, and retention of information. The first tow elements appear to involve the hippocampus and medial temporal lobe structures. The retention or storage appears to involve the heteromodal association areas. Amnesia can be experienced as a loss of stored memory or an inability to form new memories. The loss of stored memories is known as retrograde amnesia. The inability to form new memories is known as anterograde amnesia.

Complaints of memory problems are common. Poor concentration, poor arousal and poor attention all may disrupt the memory process to a degree. The subjective complaint of memory problems therefore must be distinguished from true amnesias. This is usually done at the bedside in a more gross evaluation and through specific neurophyschological tests. Defects in visual and verbal memory can be separated through such tests. In amnesias there is by definition a preservation of other mental capacities such as logic. The neurobiologic theory of memory described above would predict that amnesias would have relatively few pathobiologic variations. Clinically the problem of amnesias often appears as a result of a sudden illenss in an otherwise healthy person.

Exemplary forms of amnesias which may be treated by the subject method include amnesias of short duration, alcoholic blackouts, Wernicke-Korsakoff's (early), partial complex seizures, transient global amnesia, those which are related to medication, such as triazolam (Halcion), and basilar artery migrines. The subject method may also be used to treat amnesias of longer duration, such as post concussive or as the result of Herpes simplex encephalitis.

### Exemplification

The invention now being generally described, it will be more readily understood by reference to the following examples which are included merely for purposes of illustration of certain aspects and embodiments of the present invention, and are not intended to limit the invention.

Background Information and Objective

The Inhibitory Avoidance (IA) task is a well-studied behavioral paradigm which can provide the researcher with a consistent and long-lasting measure of memory. The paradigm consists of one training trial and one retention trial. Test articles may be administered to the rats either before or after training. Improved memory, as a result of test compound administration, is evident as increased latency on the retention trial. The objective of the following experiments was to investigate the effects of methylphenidate on IA memory in the rat.

### EXAMPLE 1

### Dose Response Testing

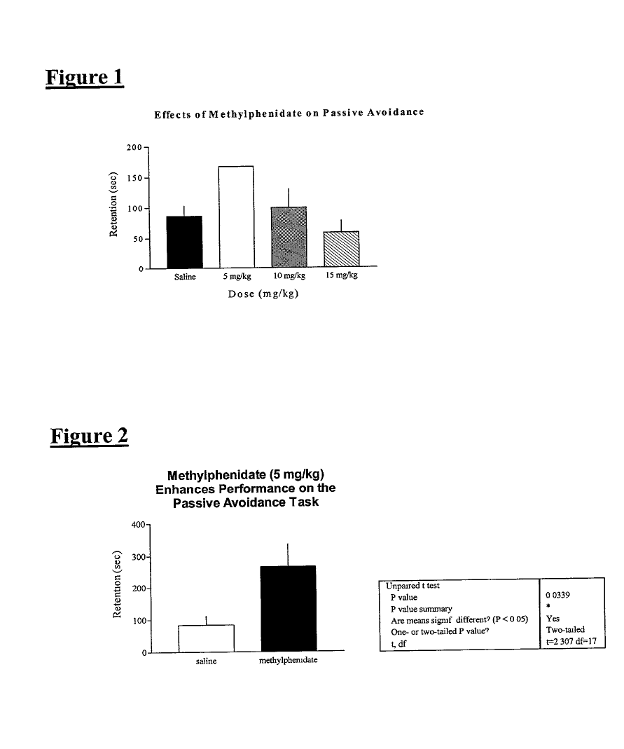

In this experiment, rats were injected with three different doses of methylphenidate thirty minutes prior to training on the IA task. As can be seen from FIG. 1, a dose of 5 mg/kg improved retention, while doses of 10 and 15 mg/kg had no effect.

In order to verify this result, a second experiment was conducted. Rats were injected with 5 mg/kg of methylphenidate and trained on the IA task. As can be seen from FIG. 2, this dose of methylphenidate significantly improved retention of the task. An unpaired t-test demonstrated that this enhancement was statistically significant (p<0.03).

### EXPERIMENT 2

### Time Course of Effectiveness

In this experiment, the time of drug administration was varied in order to determine the optimal pre-training drug administration time. This experiment demonstrated that methylphenidate (5 mg/kg) is most effective when administered to the rats one hour prior to training.

### EXPERIMENT 3

### Long-Term Retention

This experiment was conducted in order to determine whether the enhanced retention observed in Experiment 2 was long-lasting. Rats received a second retention test one week after the first retention test. No additional training or drug was administered to the animals in the interim period. Results demonstrated that performance of the methylphenidate-injected rats was still significantly enhanced one week following the original training session (t(54)=2.358,p<0.0220).

### EXPERIMENT 4

### Effects on Lesioned Rats

The findings of the above experiments are important, as they identify the most effective dose and time of administration for this compound. Moreover, the results demonstrate that methylphenidate improves memory in normal rats, and that this improvement is long lasting. In the next experiment, we investigated whether the performance of amnesic rats could be improved by administration of methylphenidate. In this experiment, control rats and rats with lesions of the fornix received injections of either saline or methylphenidate (5 mg/kg), and one hour later, were tested on the IA task.

As FIG. 3 illustrates, methylphenidate dramatically enhanced the performance of normal rats and in addition, appeared to improve the performance of the fornix lesion rats. A one-way ANOVA demonstrated that there was a significant difference between the performance of the four groups (F(3, 36)=4.497, p<0.009). Student-Newman-Keuls post hoc tests revealed firstly that the performance of normal rats that received methylphenidate was significantly enhanced relative to all other experimental groups (p<0.05). In addition, the performance of fornix animals that received methylphenidate was not significantly different from normal, saline injected animals. These results demonstrate that methylphenidate is capable of enhancing memory in normal rats and has beneficial effects in brain damaged, amnesic rats.

### EXAMPLE 5

### Effects of d and l-Threo Methylphenidate on Inhibitory Avoidance

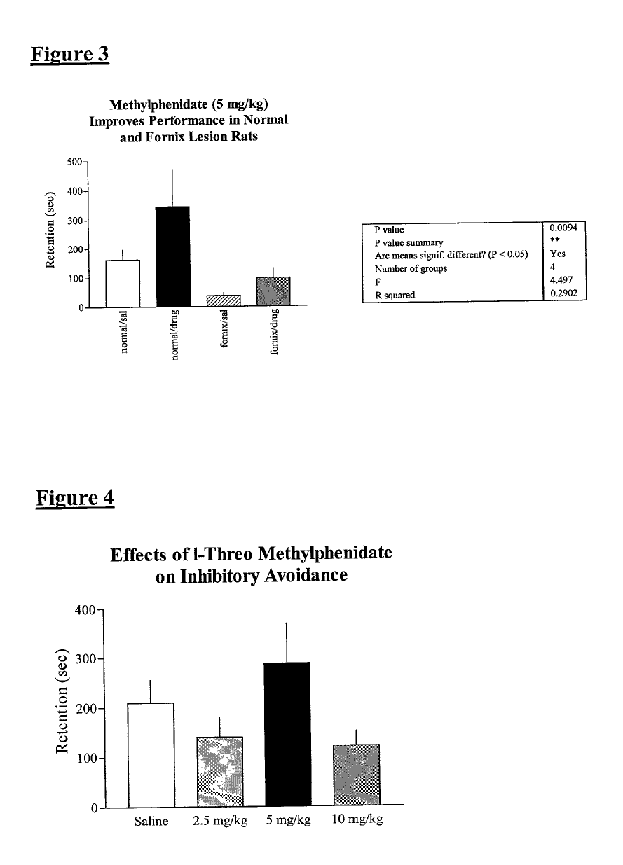

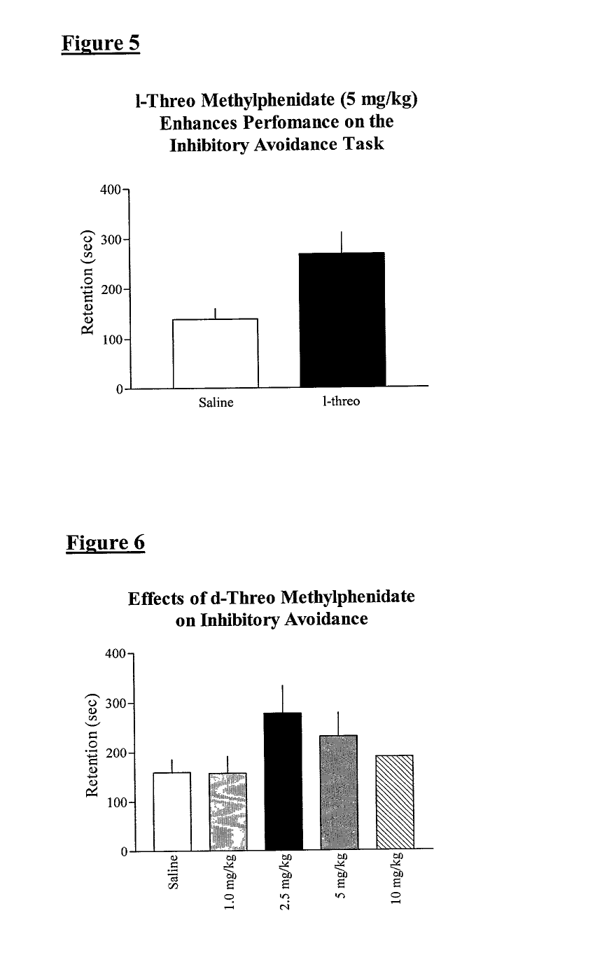

Different doses of l-threo methylphenidate were administered to rats one hour prior to training on the Inhibitory Avoidance task. Retention for the task was tested 24 hours later. As can be seen from FIG. 4, a dose of 5 mg/kg appeared to be effective in enhancing performance on this task. This experiment was subsequently replicated using a target dose of 5 mg/kg. As FIG. 5 illustrates, 5 mg/kg of l-threo methylphenidate significantly enhanced performance of the Inhibitory Avoidance task (t(59)=2.686, p<0.0094).

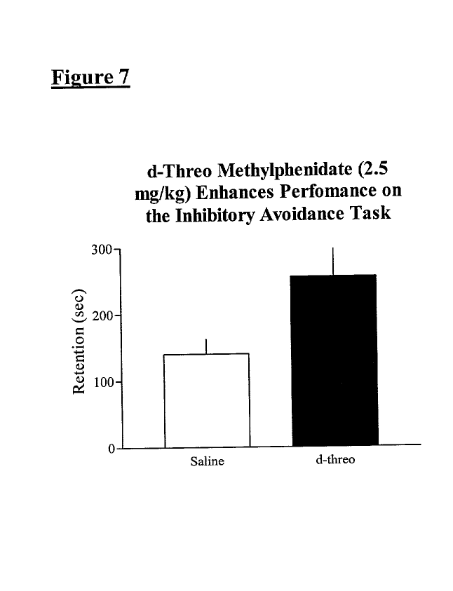

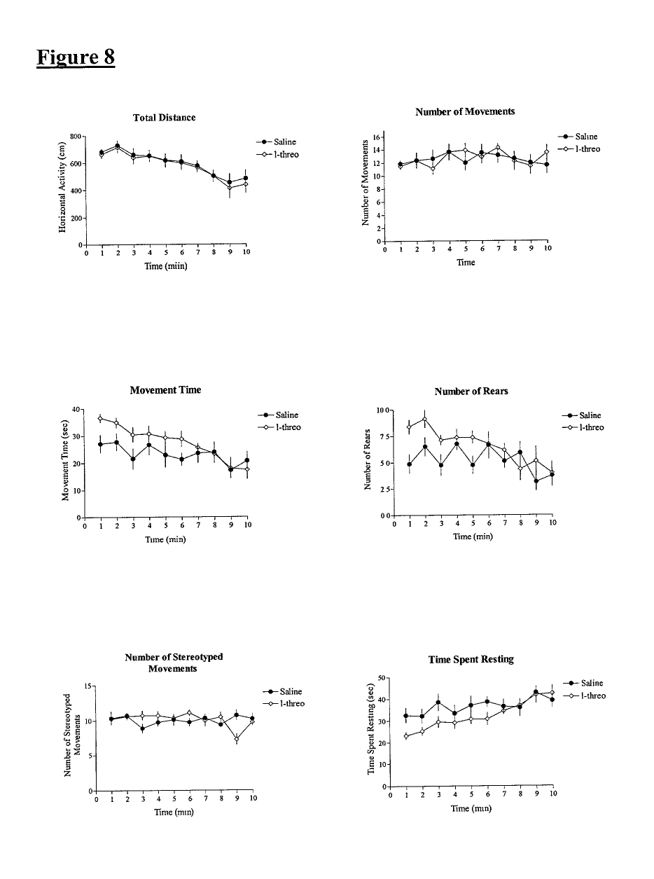

The effects of d-threo methylphenidate on memory consolidation as tested by the Inhibitory Avoidance task were also evaluated. Different doses of d-threo were administered to rats one hour prior to training on the Inhibitory Avoidance task. Results from this experiment are presented in FIG. 6, and demonstrate that 2.5 mg/kg appears to be effective in improving memory. This experiment was subsequently replicated, and results demonstrated that a dose of 2.5 mg/kg of d-threo methylphenidate has significant memory enhancing effects (t(85)=2.403, p<0.0184). These results are illustrated in FIG. 7.

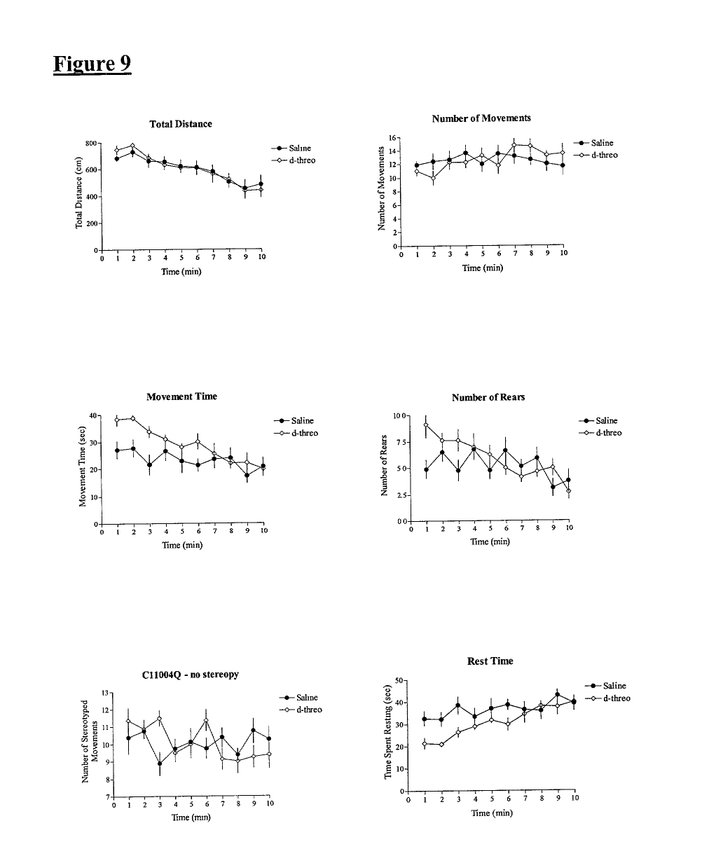

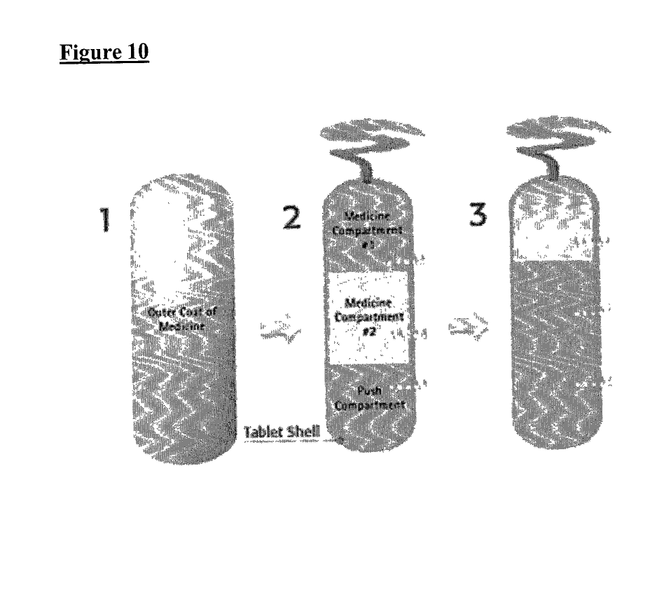

The effects of d-threo methylphenidate on locomotor activity was investigated by injecting rats with saline or 2.5 mg/kg of d-threo methylphenidate, and testing them one hour later on the activity monitoring task. Results from this experiment, presented in FIG. 9, demonstrate that like l-threo, d-threo methylphenidate did not produce a large amount of hyperactivity. No significant main effects were observed for total distance moved, number of movements or number of stereotyped activities. Significant main effects were observed for movement time (F(1, 70)=1304, p<0.0001), number of rears (F(1, 70)=19.60, p<0.0234) and rest time (F(1, 70)=1322, p<0.0001).

### EXAMPLE 6

### Effects of d and l-Threo Methylphenidate on Activity Levels

In order to investigate the effects of l-threo methylphenidate on activity levels, rats were injected with 5 mg/kg of l-threo methylphenidate, and underwent activity monitoring one hour later. Administration of l-threo methylphenidate did not result in greatly increased activity levels, as compared to saline injected controls (see FIG. 8). There was no significant difference between saline and l-threo injected animals on the measures of total distance moved, number of movements or number of stereotyped activities. There were small, but statistically significant main effects for movement time (F(1, 70)=728, p<0.0033), number of rears (F(1, 70)=74.26, p<0.0001), and time spent resting (F(1, 70)=730.6, p<0.0032). Overall, administration of l-threo methylphenidate does not appear to result in serious locomotor hyperactivity.

### EXAMPLE 7

### A Randomised, Double-Blind, Placebo-Controlled Study Of l-Threo Methylphenidate Hydrochloride in Healthy, Normal Human Volunteers

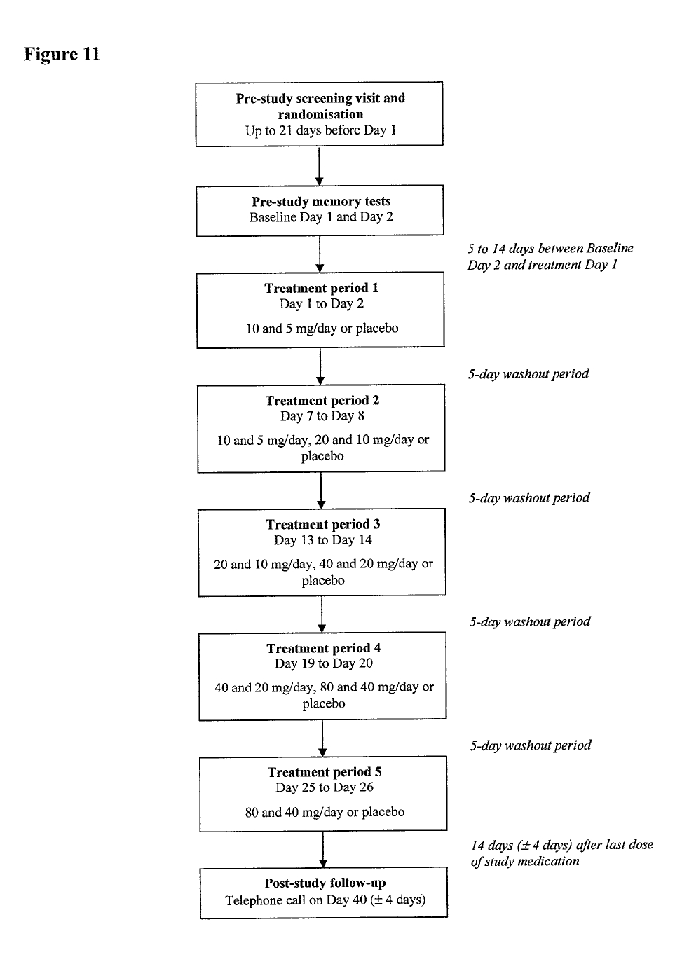

A schematic illustration of the study design, detailing the timing of visits and randomisation is shown in FIG. 11. The primary objective of the study is to identify the MTD and dose-limiting side-effects of l-threo methylphenidate hydrochloride. The secondary objectives included:

To assess the effects of l-threo methylphenidate on quantitative memory scores

To assess the perceived CNS effects following l-threo methylphenidate administration

To assess the effects of l-threo methylphenidate on the cardiovascular system

To explore the relationship between dose, tolerability, safety and pharmacological effects of l-threo methylphenidate.

To define the pharmacokinetics (PK) of l-threo methylphenidate.

A minimum of four and up to eight healthy adult male/female subjects were required to take part in the study. Subjects weree selected from the volunteer database at PPD Development Clinic according to the study entry criteria. Study-related procedures were carried out before informed consent had been given. All information provided to the volunteers, including advertisements, will be approved by the Independent Ethics Committee (IEC) before use.

This was a randomised, double-blind, placebo-controlled, study including eight healthy male/female subjects. There were five treatment periods, each comprising two treatment days, with a 5-day washout period between dose levels. Subjects received four ascending doses of drug (a split dose on the first day of the treatment period and half the dose on the second day of the treatment period) and placebo. The placebo dose was randomly assigned to one of the five treatment periods. Each dose was taken orally.

The first dose level was administered on Day 1 and Day 2. After a 5-day washout period, subjects received the next dose level for 2 days. The period of washout and dose escalation was repeated until all five dose levels (four active dose levels and one placebo) were administered. Safety and tolerability was assessed after each treatment period to determine if subjects may advance to the next dose level.

Each subject received placebo, thus providing a within-subject control. A washout period of 5 days was considered sufficient to allow elimination of l-threo methylphenidate, as dl-threo methylphenidate has a half-life of no more than 2 to 3 hours in adults and is excreted within 48 to 96 hours of dosing, with strong evidence of an even shorter half-life of l-threo methylphenidate.

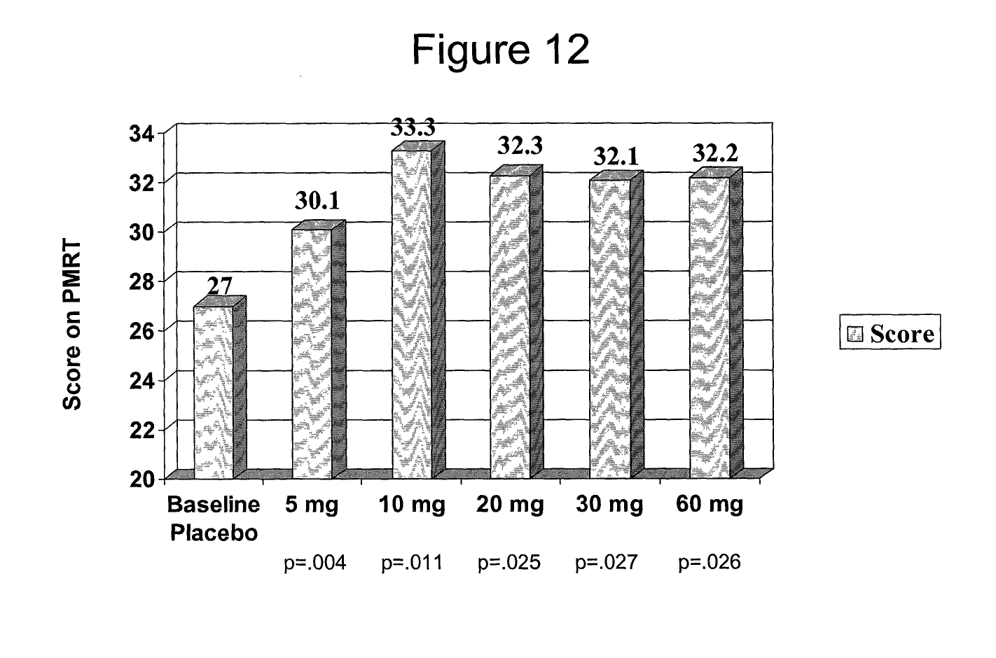

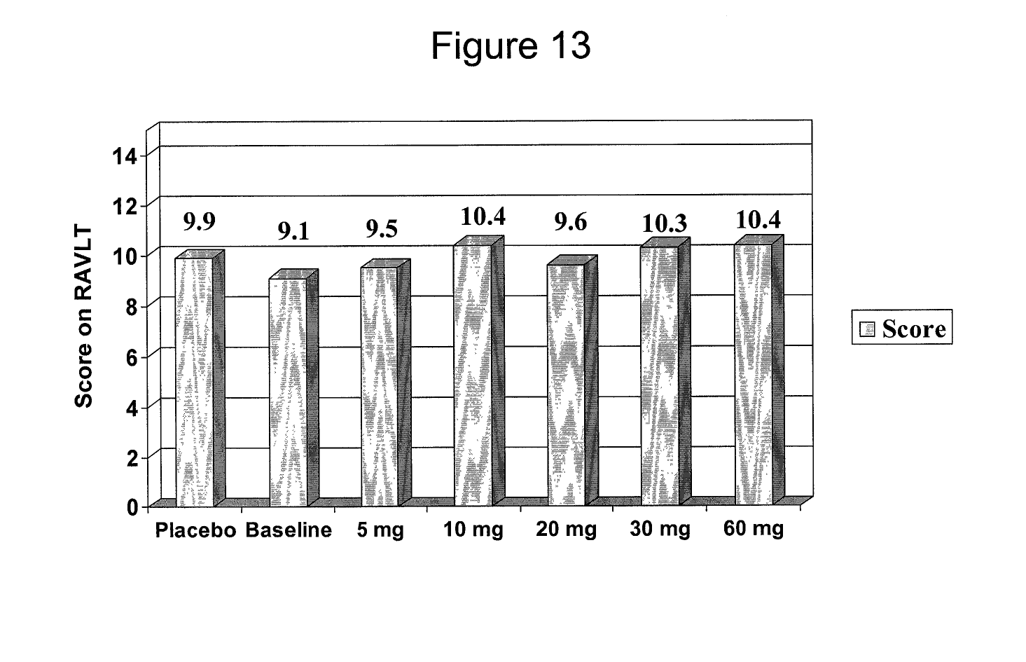

FIG. 12 shows memory retention at 60 minutes during ascending blood concentration of l-threo-methylphenidate. Patients were assessed using the Providence Recognition Memory Test (Pictoral). Higher PRMT score indicates increased memory retention. FIG. 13 shows the consequence of l-threo-methylphenidate on patient scoring using the Rey Auditory & Visual Learning Test (30 minutes). This study was performed on Decending Plasma Curve of l-threo-methylphenidate. Higher RAVLT score indicates increased memory retention. These two experiments demonstrate that l-threo-methylphenidate can enhance memory in patients, and is more effective during the ascending portion of the plasma curve.

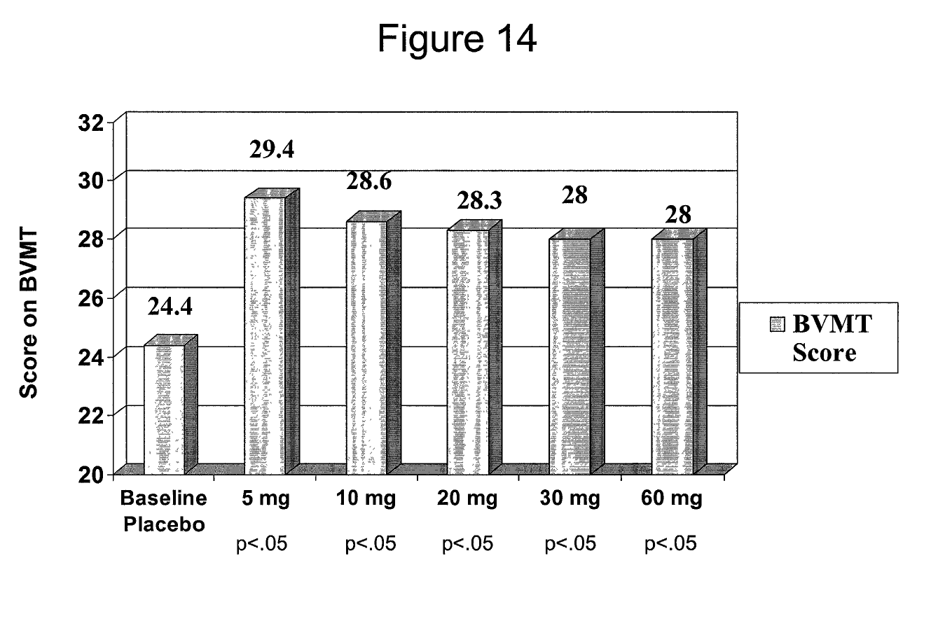

FIG. 14 shows the results of l-threo-methylphenidate on attention and learning as measured by the Brief Visuospatial Memory Test during ascending blood concentration of l-Threo-Methylphenidate. Higher BVMT score indicates increased attention and learning. This experiment demonstrate that l-threo-methylphenidate can enhance attention and learning in patients, and is more effective during the ascending portion of the plasma curve.

Equivalents

Those skilled in the art will recognize, or be able to ascertain using no more than routine experimentation, many equivalents to the specific embodiments of the invention described herein. Such equivalents are intended to be encompassed by the following claims.

All patents, publications, and other references cited above are hereby incorporated by reference in their entirety.

We claim:

1. A method for enhancing memory in an animal, comprising administering to the animal a formulation of one or more methylphenidate compounds, or pharmaceutically acceptable derivative, salt, solvate, pro-drug or metabolic derivative thereof, in an amount sufficient to enhance long-term memory in the animal, wherein the formulation includes at least 60 mol percent of a eutomer(s) relative to the distomer(s) of the methylphenidate compound(s).

2. The method of claim 1, wherein the formulation includes at least 60 percent (w/w) of a eutomer(s) of methylphenidate compound(s) represented by the general formula: 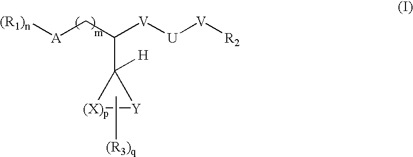
wherein
A represents a carbocyclic, heterocyclic, aryl, or heteroaryl ring;
U is absent or represents —C(═O)—, —C(═S)—, —P(═O)(OR₈)—, —S(O₂)—, or —S(O)—;
V, independently for each occurrence, is absent or represents NR, O, or S;
Y represents NR₄, O, or S;
each occurrence of X, independently, is an atom selected from C, N, S, Se, and O;
R, independently for each occurrence, represents H, lower alkyl, lower alkenyl, aryl, heteroaryl, aralkyl, or heteroaralkyl;
each occurrence of R₁ represents, independently, aryl, C1-C6 alkyl, C1-C6 alkoxy, C1-C6 acyloxy, hydroxyl, C1-C6 alkanoyl, halogen, cyano, carboxyl, amido, amino, C1-C6 acylamino, C1-C6 alkylamino, nitro, sulfonic acid, or sulfhydryl;
R₂ is selected from H, C1-C6 alkyl, and C1-C6 alkanoyl;
R₃ represents, independently for each occurrence, hydrogen, C1-C6 alkyl, C1-C6 alkoxy, hydroxyl, C1-C6 alkanoyl, halogen, carboxyl, C2-C6 alkanoxy, nitro, or sulfhydryl, or two of R₃, taken together, represent an oxo group or a double bond between two adjacent X atoms;
R₄ represents hydrogen, lower alkyl, acyl, aryl, aralkyl, heteroaryl, or heteroaralkyl, preferably hydrogen or lower alkyl;
R₈ represents hydrogen, or a substituted or unsubstituted alkyl, alkenyl, aryl, aralkyl, cycloalkyl, cycloalkenyl, or heterocycle;
m is an integer selected from 0 and 1;
n is an integer from 0 to 7;
p is an integer selected from 3, 4, 5, and 6; and
q is an integer from 0 to 16; or
a pharmaceutically acceptable derivative, salt, solvate, pro-drug or metabolic derivative thereof.

3. A method for enhancing memory in an animal, comprising administering to the animal a formulation of a methylphenidate compound, or pharmaceutically acceptable derivative, salt, solvate, pro-drug or metabolic derivative thereof, in an amount sufficient to enhance long-term memory in the animal, wherein the formulation includes at least 60 percent (w/w) of the eutomer(s) relative to the distomer(s) of the methylphenidate compound(s).

4. The method of claim 3, wherein the eutomer of the methylphenidate compound is a compound represented in the general formula (IA), or pharmaceutically acceptable salt, pro-drug or metabolic derivative thereof: 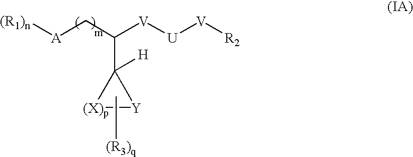
wherein
A represents a carbocyclic, heterocyclic, aryl, or heteroaryl ring;
U is absent or represents —C(═O)—, —C(═S)—, —P(═O)(OR₈)—, —S(O₂)—, or —S(O)—;
V, independently for each occurrence, is absent or represents NR, O, or S;
Y represents NR₄, O, or S;
each occurrence of X, independently, is an atom selected from C, N, S, Se, and O;
R, independently for each occurrence, represents H, lower alkyl, lower alkenyl, aryl, heteroaryl, aralkyl, or heteroaralkyl;
each occurrence of R₁ represents, independently, aryl, C1-C6 alkyl, C1-C6 alkoxy, C1-C6 acyloxy, hydroxyl, C1-C6 alkanoyl, halogen, cyano, carboxyl, amido, amino, C1-C6 acylamino, C1-C6 alkylamino, nitro, sulfonic acid, or sulfhydryl;
R₂ is selected from H, C1-C6 alkyl, and C1-C6 alkanoyl;
R₃ represents, independently for each occurrence, hydrogen, C1-C6 alkyl, C1-C6 alkoxy, hydroxyl, C1-C6 alkanoyl, halogen, carboxyl, C2-C6 alkanoxy, nitro, or sulfhydryl, or two of R₃, taken together, represent an oxo group or a double bond between two adjacent X atoms;
R₄ represents hydrogen, lower alkyl, acyl, aryl, aralkyl, heteroaryl, or heteroaralkyl, preferably hydrogen or lower alkyl;
m is an integer selected from 0 and 1;
n is an integer from 0 to 7;
p is an integer selected from 3, 4, 5, and 6; and
q is an integer from 0 to 16, or
a pharmaceutically acceptable derivative, salt, solvate, pro-drug or metabolic derivative thereof.

5. The method of claim 3, wherein the eutomer of the methylphenidate compound is represented in the general formula (II), or pharmaceutically acceptable salt, pro-drug or metabolic derivative thereof: 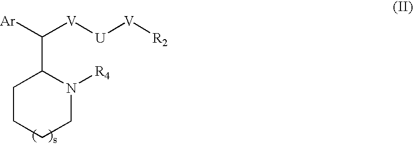
wherein
U is absent or represents —C(═O)—, —C(═S)—, —P(═O)(OR₈)—, —S(O₂)—, or —S(O)—;
V, independently for each occurrence, is absent or represents NR, O, or S;
R, independently for each occurrence, represents H, lower alkyl, lower alkenyl, aryl, heteroaryl, aralkyl, or heteroaralkyl;
R₂ is selected from H, C1-C6 alkyl, and C1-C6 alkanoyl;
R₄ represents hydrogen, lower alkyl, acyl, aryl, aralkyl, heteroaryl, or heteroaralkyl, preferably hydrogen or lower alkyl;
s represents an integer from 0 to 2; and
Ar represents a substituted or unsubstituted aryl or heteroaryl group.

6. The method of claim 3, wherein the pharmaceutically acceptable salt of the eutomer of the methylphenidate compound has a structrure represented in the general formula (III): 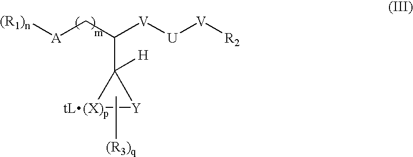
wherein
A represents a carbocyclic, heterocyclic, aryl, or heteroaryl ring;
U is absent or represents —C(═O)—, —C(═S)—, —P(═O)(OR₈)—, —S(O₂)—, or —S(O)—;
V, independently for each occurrence, is absent or represents NR, O, or S;
Y represents NR₄, O, or S;
each occurrence of X, independently, is an atom selected from C, N, S, Se, and O;
R, independently for each occurrence, represents H, lower alkyl, lower alkenyl, aryl, heteroaryl, aralkyl, or heteroaralkyl;
each occurrence of R₁ represents, independently, aryl, C1-C6 alkyl, C1-C6 alkoxy, C1-C6 acyloxy, hydroxyl, C1-C6 alkanoyl, halogen, cyano, carboxyl, amido, amino, C1-C6 acylamino, C1-C6 alkylamino, nitro, sulfonic acid, or sulfhydryl;
R₂ is selected from H, C1-C6 alkyl, and C1-C6 alkanoyl;
R₃ represents, independently for each occurrence, hydrogen, C1-C6 alkyl, C1-C6 alkoxy, hydroxyl, C1-C6 alkanoyl, halogen, carboxyl, C2-C6 alkanoxy, nitro, or sulfhydryl, or two of R₃, taken together, represent an oxo group or a double bond between two adjacent X atoms;
R₄ represents hydrogen, lower alkyl, acyl, amido, ester, aryl, aralkyl, heteroaryl, or heteroaralkyl, preferably hydrogen or lower alkyl;
m is an integer selected from 0 and 1;
n is an integer from 0 to 7;
p is an integer selected from 3, 4, 5, and 6; and
q is an integer from 0 to 16;
L is a non-toxic organic or inorganic acid, or a quatemizing agent, or any combination thereof; and
t is an integer from 1 to 6.

7. The method of claim 3, wherein the pharmaceutically acceptable salt of the eutomer of the methylphenidate compound has a structrure represented in the general formula (IV), or a pharmaceutically acceptable salt, solvate or pro-drug thereof. 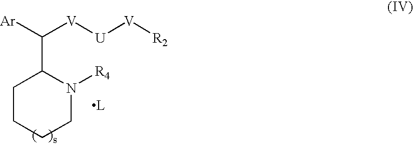
wherein
U is absent or represents —C(═O)—, —C(═S)—, —P(═O)(OR₈)—, —S(O₂)—, or —S(O)—;
V, independently for each occurrence, is absent or represents NR, O, or S;
R, independently for each occurrence, represents H, lower alkyl, lower alkenyl, aryl, heteroary, aralkyl, or heteroaralkyl;
R₂ is selected from H, C1-C6 alkyl, and C1-C6 alkanoyl;
R₄ represents hydrogen, lower alkyl, acyl, aryl, aralkyl, heteroaryl, or heteroaralkyl, preferably hydrogen or lower alkyl;
s represents an integer from 0 to 2;
Ar represents a substituted or unsubstituted aryl or heteroaryl group; and
L is a non-toxic organic or inorganic acid, or a quatemizing agent, or any combination thereof.

8. The method of claim 3, wherein the metabolite of the eutomer of the methylphenidate compound has a structrure represented in the general formula (V), or a pharmaceutically acceptable salt, solvate or pro-drug thereof: 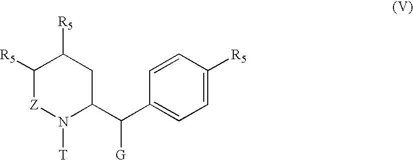
wherein
R₅, independently for each occurrence, is absent or represents hydroxyl;
Z represents —CH₂— or —C(═O)—;
T represents hydrogen or —C(═O)—NH₂;
G represents carboxylic acid, or a pharmaceutically acceptable salt thereof, carboxylic acid methyl ester, carboxylic acid ethyl ester, or acetylamino ethane sulfonic acid.

9. The method of any of claims 4, 5, 6, 7 or 8, wherein R₂ represents H or C1-C6 alkyl.

10. The method of any of claims 4, 5, 6, 7 or 8, wherein U represents —(═O)— or —C(═S)—.

11. The method of any of claims 4, 5, 6, 7 or 8, wherein at least one occurrence of V is present.

12. The method of claim 11, wherein V is absent for one occurrence, and in the other V represents NH, S, or O.

13. The method of claim 4 or 6, wherein A represents an aryl or heteroaryl group.

14. A pharmaceutical preparation comprising a methylphenidate compound in an amount sufficient to enhance long-term memory in the animal, wherein the preparation includes at least 60 percent (w/w) of the eutomer(s) relative to the distomer(s) of the methylphenidate compound(s).

15. A single dosage pharmaceutical preparation for oral administration to a human patient, comprising one or more methylphenidate compounds in an amount sufficient to enhance long-term memory in the animal, wherein the preparation includes at least 60 percent (w/w) of the eutomer(s) relative to the distomer(s) of the methylphenidate compound(s), and is formulated for delivering a sustained and increasing dose over at least 4 hours to lessen the incidence of tolerance in the patient.

16. The preparation of claim 15, for delivering a sustained and increasing dose over at least 8 hours.

17. The preparation of claim 15, comprising a multiplicity of layers each including the same or different polymers, a dose of the methylphenidate compound(s) in an increasing dose in the multiplicity of layers, wherein in operation the preparation delivers an increasing dose of the methylphenidate compound(s) over time.

18. The preparation of claim 15, comprising a bioerodible polymer, a dose of the methylphenidate compound(s) present in an initial dose and a final dose, whereby the preparation delivers an initial dose then a final dose over time.

19. The preparation of claim 15, comprising a plurality of beads, each bead including a methylphenidate compound and having a dissolution profile, which plurality of beads is a variegated population with respect to dose and/or dissolution profile so as deliver, upon administration, said sustained and increasing dose over at least 4 hours

20. The preparation of claim 15, wherein the methylphenidate compound is (i) contained within a nonabsorbable shell that releases the drug at a controlled rate, and (i) formulated in at least two different dissolution profiles.

21. A kit comprising:
a. a pharmaceutical preparation comprising a methylphenidate compound in an amount sufficient to enhance long-term memory in the animal, wherein the preparation includes at least 60 percent (w/w) of a eutomer(s) relative to a distorner(s) of the methylphenidate compound(s); and
b. instructions, written and/or pictorial, describing the use of the preparation for enhancing memory in a patient.

22. The kit of claim 21, wherein the methylphenidate compound is provided in single dosage form.

23. The kit of claim 22, wherein the methylphenidate compound is formulated for delivering a sustained and increasing dose over at least 4 hours to lessen the incidence of tolerance in the patient.

24. The kit of claim 21, wherein the methylphenidate compound is provided in the form of a transdermal patch.

25. The method of claim 1 or 3, the preparation of claim 14 or 15 or the kit of claim 21, wherein said methylphenidate compound(s) is provided in an amount sufficient to enhance long-term memory in a patient by a statistically significant amount when assessed by a standardized performance test.

26. The method, preparation or kit of claim 25, wherein said standardized test is selected from the group consisting of: Cambridge Neuropsychological Test Automated Battery (CANTAB); a Children's Memory Scale (CMS); a Contextual Memory Test; a Continuous Recognition Memory Test (CMRT); a Denman Neuropsychology Memory Scale; a Fuld Object Memory Evaluation (FOME); a Graham-Kendall Memory for Designs Test; a Guild Memory Test; a Learning and Memory Battery (LAMB); Rey Auditory and Verbal learning Test (RAVLT); Brief Visuospatial Memory Test (BVMT); Providence Recognition Memory Test (PRMT), a Memory Assessment Clinic Self-Rating Scale (MAC-S); a Memory Assessment Scales (MAS); a Randt Memory Test; a Recognition Memory Test (RMT); a Rivermead Behavioral Memory Test; a Russell's Version of the Wechsler Memory Scale (RWMS); a Test of Memory and Learning (TOMAL); a Vermont Memory Scale (VMS); a Wechsler Memory Scale; and a Wide Range Assessment of Memory and Learning (WRAML).

27. The method, preparation or kit of claim 25, wherein said standardized test is a Providence Recognition Memory Test.

28. The method of claim 1 or 3, further comprising administering one or more of a neuronal growth factor, a neuronal survival factor, a neuronal trophic factor, a cholinergic modulator, an adrenergic modulator, a nonadrenergic modulator, a dopaminergic modulator, a glutaminergic modulator or an agent that stimulates the PKC, PKA, GABA, NMDA, cannabinoid, AMPA, kainate, phosphodiesterase (PDE), CREB or nootropic pathways.

29. The method of claim 1 or 3, the preparation of claim 14 or 15 or the kit of claim 21, for use in the prophylaxis or treatment of a patient susceptible to or suffering from memory impairment.

30. The method, preparation or kit of claim 29, for use in the prophylaxis or treatment of a patient susceptible to or suffering from impaired memory due to anxiety, depression, age-associated memory impairment, minimal cognitive impairment, amnesia, dementia, learning disabilities, memory impairment associated with toxicant exposure, brain injury, brain aneurysm, Parkinson's disease, head trauma, Huntington's disease, Pick's disease, Creutzfeldt-Jakob disease, stroke, schizophrenia, epilepsy, mental retardation, Alzheimer's disease, age, attention deficit disorder, attention deficit hyperactivity disorder, AIDS-related dementia, sleep deprivation, a sleep disorder.

31. The method of claim 1 or 3, the preparation of claim 14 or 15 or the kit of claim 21, for enhancing memory in normal individuals.

32. A method for conducting a pharmaceutical business, comprising:
a. manufacturing a preparation of claims 14 or 15; and
b. marketing to healthcare providers the benefits of using the preparation to increase memory function.

33. A method for conducting a pharmaceutical business, comprising:
a. providing a distribution network for selling the preparation of claims 14 or 15; and
b. providing instruction material to patients or physicians for using the preparation to increase memory function.

34. A method for conducting a pharmaceutical business, comprising:
a. determining an appropriate preparation and dosage of a methylphenidate compound to increase memory function;
b. conducting therapeutic profiling of preparations identified in step (a), for efficacy and toxicity in animals; and
c. providing a distribution network for selling a preparation identified in step (b) as having an acceptable therapeutic profile.

35. The method of claim 34, including an additional step of providing a sales group for marketing the preparation to healthcare providers.

36. A method for conducting a pharmaceutical business, comprising:
a. determining an appropriate preparation and dosage of methylphenidate to be administered to increase memory function; and
b. licensing, to a third party, the rights for further development and sale of the preparation.37. Specific treatment for ADD.

37. A method for enhancing attention span in an animal, comprising administering to the animal a formulation of one or more methylphenidate compounds, or pharmaceutically acceptable derivative, salt, solvate, pro-drug or metabolic derivative thereof, in an amount sufficient to enhance attention span in the animal, wherein the formulation includes at least 60 percent (w/w) of a L-threo (2S:2′S) stereoisomer, a D-threo (2R:2′R) stereoisomer, or a combination thereof of the methylphenidate compound relative to erythro-isomers of the methylphenidate compound.

38. The method of claim 37, wherein the animal has a condition characterized by a deficit in attention span.

39. The method of claim 38, wherein the condition is selected from the group consisting of Attention Deficit Disorder, Attention Deficit Disorder with Hyperactivity, Tourette's Syndrome, autism, depression, and bi-polar disorder.

40. The method of claim 38, wherein the condition is selected is selected from the group consisting of Attention Deficit Disorder and Attention Deficit Disorder.

41. A method for Attention Deficit Disorder (ADD) or Attention Deficit Disorder with Hyperactivity (ADHD) in an animal, comprising administering to the animal a formulation of one or more methylphenidate compounds, or pharmaceutically acceptable derivative, salt, solvate, pro-drug or metabolic derivative thereof, wherein the formulation includes at least 60 percent (w/w) of a L-threo (2S:2′S) stereoisomer, a D-threo (2R:2′R) stereoisomer, or a combination thereof of the methylphenidate compound relative to erythro-isomers of the methylphenidate compound.

42. A method of enhancing attention span in a patient, comprising administering the pharmaceutical preparation of claim 14 or 15.

43. A method for treating a condition characterized by a deficit in attention span, comprising administering the pharmaceutical preparation of claim 14 or 15.

44. A single dose formulation of one or more methylphenidate compounds, wherein the single dose is greater than 60 mg.

44. The formulation of claim 43, wherein the single dose is greater than 100 mg.

45. The formulation of claim 44, wherein the single dose is greater than 250 mg.

46. The formulation of claim 45, wherein the single dose is greater than 500 mg.

47. The method of claim 3, wherein the formulation includes at least at least 60 percent (w/w) of a L-threo (2S:2′S) stereoisomer of methylphenidate relative to D-threo (2R:2′R), D-erythro (2R:2′S) and L-erythro (2S:2′R) isomers of methylphenidate.

48. The pharmaceutical preparation of claim 14, wherein the preparation includes at least 60 percent (w/w) of a L-threo (2S:2′S) stereoisomer, a D-threo (2R:2′R) stereoisomer, or a combination thereof of methylphenidate relative to erythro-isomers of methylphenidate.

49. The single dosage pharmaceutical preparation of claim 15, wherein the preparation includes at least 60 percent (w/w) of a L-threo (2S:2′S) stereoisomer, a D-threo (2R:2′R) stereoisomer, or a combination thereof of methylphenidate relative to erythro-isomers of methylphenidate.

50. The kit of claim 21, wherein the preparation includes at least 60 percent (w/w) of a L-threo (2S:2′S) stereoisomer, a D-threo (2R:2′R) stereoisomer, or a combination thereof of methylphenidate relative to erythro-isomers of methylphenidate.
